(19) United States
(12) Patent Application Publication
(10) Pub. No.: US 2024/0001053 A1
(43) Pub. Date: Jan. 4, 2024

(54) ELECTRO-IONIC SYSTEMS AND METHODS FOR TREATING ENCLOSED SPACES AND MEDICAL AIR AND GAS SUPPLY DEVICES FOR IMPROVED PROTECTION FROM AIRBORNE BIOPATHOGENS

(71) Applicant: Julian HENLEY, New Orleans, LA (US)

(72) Inventor: Julian HENLEY, New Orleans, LA (US)

(21) Appl. No.: 18/368,470

(22) Filed: Sep. 14, 2023

Related U.S. Application Data
(63) Continuation of application No. PCT/US2022/071169, filed on Mar. 15, 2022.
Application No. PCT/US2022/071169 is a continuation-in-part of application No. PCT/US2021/022392, filed on Mar. 15, 2021.
(60) Provisional application No. 62/988,991, filed on Mar. 13, 2020.
(60) Provisional application No. 63/027,746, filed on May 20, 2020.
(60) Provisional application No. 63/043,424, filed on Jun. 24, 2020.
(60) Provisional application No. 63/044,768, filed on Jun. 26, 2020.
(60) Provisional application No. 63/063,968, filed on Aug. 11, 2020.
(60) Provisional application No. 63/113,598, filed on Nov. 13, 2020.
(60) Provisional application No. 63/202,022, filed on May 24, 2021.
(60) Provisional application No. 63/226,550, filed on Jul. 28, 2021.
(60) Provisional application No. 63/310,827, filed on Feb. 16, 2022.
(60) Provisional application No. 63/310,842, filed on Feb. 16, 2022.

(51) Int. Cl. A61M 13/00; A61M 99/00; C01B 13/11; A61L 9/22; A61L 9/015

(52) U.S. Cl. CPC A61M 13/003; A61M 99/00; C01B 13/115; A61L 9/22; A61L 9/015; A61M 2205/3334; A61M 2202/0216; A61M 2202/0208; A61M 2202/0007; A61M 2202/0014; A61M 2205/3327; C01B 2201/12; C01B 2201/24; A61L 2202/10; A61L 2202/20

(57) ABSTRACT

Aspects of the present disclosure include a medical insufflation device for use on a patient body. The device includes a chamber, an ozone generator, an instrument, and a controller. The chamber is configured to receive a medical gas at least including oxygen. The ozone generator is in communication with the medical gas and configured to generate an ozonated medical gas by converting at least a portion of the oxygen in the medical gas into ozone. The instrument is configured to be introduced into the patient body. Further, the instrument is also configured to receive the ozonated medical gas from the chamber and convey the ozonated medical gas into the patient body. The controller is configured to control the device such that the ozonated medical gas conveyed to the patient body by the instrument is at a targeted amount of ozone.

### CROSS REFERENCE TO RELATED APPLICATION

This application claims priority from Intl Appln. No. PCT/US2021/022392 filed on Mar. 15, 2021, U.S. Provisional Patent Appln. No. 62/988,991 filed on Mar. 13, 2020, U.S. Provisional Patent Appln. No. 63/027,746 filed on May 20, 2020, U.S. Provisional Patent Appln. No. 63/043,424 filed on Jun. 24, 2020, U.S. Provisional Patent Appln. No. 63/044,768 filed on Jun. 26, 2020, U.S. Provisional Patent Appln. No. 63/063,968 filed on Aug. 11, 2020, U.S. Provisional Patent Appln. No. 63/113,598 filed on Nov. 13, 2020, U.S. Provisional Patent Appln. No. 63/202,022 filed on May 24, 2021, U.S. Provisional Patent Appln. No. 63/226,550 filed on Jul. 28, 2021, U.S. Provisional Patent Appln. No. 63/310,827 filed on Feb. 16, 2022, and U.S. Provisional Patent Appln. No. 63/310,842 filed on Feb. 16, 2022, the entirety of each is incorporated by reference herein. This application also incorporates by reference in its entirety U.S. Pat. No. 6,901,930 filed on Oct. 28, 2002.

### FIELD OF THE INVENTION

This application relates to devices and methods for improved protection from airborne biopathogens. In particular, this application relates to systems and methods for particle capture and deactivation in enclosed spaces including, for example, but not limitation, heating, ventilation, and air conditioning (HVAC) systems, refrigerators, elevators and medical air and gas supply systems.

### BACKGROUND OF THE INVENTION

It is difficult for patients and practitioners to control the transmission of airborne viruses and infections. Examples of such infections include seasonal flu, common colds, and measles, among others. Recently, COVID-19 is thought to have a component of airborne transmission and cross infection. Some researchers believe that under normal circumstances, when small airborne particles enter the lungs, some of them may directly bypass the airway defensive system which is made up of mucous membranes in the nasal and oral cavity as well as the bronchial tree. These particles may enter the distal alveolus where they can rapidly begin contacting cells of the internal organ. Such penetration of the distal alveolus is thought to be confined to the smaller particles as the larger particles are trapped by the body's own filtration system.

Although the exact mechanism of viral transmission remains a point of controversy, some investigators lean towards the fact that viral transmission occurs through touching and then movement of the fingers to enter mucous membranes where the virus can implant itself. This theory is based on the idea that the human cough sprays larger droplets that can be effectively precipitated or filtered and do not necessarily need to be inhaled. The exact mechanism of transmission remains controversial, but some investigators postulate that the small particles penetrating the distant alveolus is a significant modality of transmission. It is quite possible that the salivary droplets and mucous droplets that contain the virus and exit an infected patient as a cough mist partially evaporate or settle onto a surface. Such micro-droplets get smaller via evaporation and may become airborne again in the proximity of the enclosed space or circulating HVAC systems, such as in buildings, automobiles, and airplanes. The airborne transmissibility is predicated on the functional viability of the virus outside of the body in the air, and in the HVAC systems. Recent studies of COVID-19 have demonstrated that the viral particle remains viable in contact with plastic or metal surfaces for extended periods of time, for hours and even days. This is worrisome because it implicates existing ventilation systems with possible spread of COVID-19 virus among other viral, bacterial, or fungal particles.

Viral transmission is known to occur in elevators, where people are confined to small spaces and there is high traffic. This creates a high potential not only for respiratory transmission, but also for direct contact since residual droplets may be retained on surfaces for extended period of time and people riding in the elevator are unavoidably within close proximity to these surfaces.

Given the challenges associated with limiting the transmission of airborne viruses and infections and the desire to reinstate the economic systems, aspects of the present disclosure were developed to provide adequate entrapment of viral particles and droplets, and to provide a virus kill technology in real-time in HVAC systems.

In addition, recent studies have shown that patients undergoing elective procedures may contain live virus despite having a negative COVID-19 swab test prior to such procedures. In particular, one study suggests residual concentration of COVID-19 in the cecum (junction of small intestine and colon) remains a reservoir for the virus even many weeks after the upper respiratory swab test has turned negative in recuperating patients. Presence of live virus and its potential for transmission in patients deemed virus free by current swab test poses a procedural risk to staff and facilities performing outpatient common procedures such as colonoscopies.

In the case of lower GI procedures, it is becoming evident that there may be a residual body reservoir of the virus for many weeks after the swab test has defined the patient as being virus free. Moreover, a previously infected patient may also have virus present in the upper GI tract, abdominal cavity, etc. In addition to COVID-19, the patient may have other pathogens present, such as C. difficile, HIV, CMV, MERSA, and staph aureus among others. Upon release of insufflation gas from an infected or recently infected patient, the clinicians and staff within the room may be exposed to the released viruses. It is desirable to reduce the viral concentration in any anatomical cavity, including the lower GI tract prior to any potential exposure to that anatomical cavity during such procedures.

### SUMMARY OF THE INVENTION

Aspects of the present disclosure include a medical insufflation device for use on a patient body. The device includes a chamber, an ozone generator, an instrument, and a controller. The chamber is configured to receive a medical gas at least including oxygen. The ozone generator is in communication with the medical gas and configured to generate an ozonated medical gas by converting at least a portion of the oxygen in the medical gas into ozone. The instrument is configured to be introduced into the patient body. Further, the instrument is also configured to receive the ozonated medical gas from the chamber and convey the ozonated medical gas into the patient body. The controller is configured to control the device such that the ozonated medical gas conveyed to the patient body by the instrument is at a targeted amount of ozone.

In one version of the medical insufflation device, the targeted amount of ozone may be any of the following, depending on a user selected or manufacture set setting: below 0.1 ppm; between 0.1 and 0.15 ppm; between 0.15 and 0.2 ppm; and/or above 0.2 ppm.

In one version of the medical insufflation device, the instrument includes at least one of a viewing instrument facilitating internal viewing within the patient body, a medical instrument for performing a surgical procedure, or a channel through which surgery is performed.

In one version of the medical insufflation device, at least a portion of the ozone generator is located in the chamber.

In a first version of the medical insufflation device, the device further includes a pressure sensor configured to measure a gas pressure at least associated with the ozonated medical gas, an ozone sensor configured to measure an ozone concentration of the ozonated medical gas, a controller configured to control the device based on measured values from the pressure sensor and ozone sensor such that the ozonated medical gas conveyed to the patient body by the instrument is at a targeted amount of ozone.

In a second version of the medical insufflation device, the device further includes a pressure sensor configured to measure a gas pressure of the ozonated medical gas in the chamber, an ozone sensor configured to measure an ozone concentration of the ozonated medical gas in the chamber, a first solenoid configured to regulate a flow of the medical gas entering the chamber, a second solenoid configured to regulate a flow of the ozonated gas exiting the chamber; and the controller is configured to control at least the first and second solenoids based on measured values from the pressure sensor and ozone sensor to provide a targeted amount of ozone leaving the chamber through the second solenoid.

In the first or second version of the medical insufflation device, the targeted amount of ozone may be any of the following, depending on a user selected or manufacture set setting: below 0.1 ppm; between 0.1 and 0.15 ppm; between 0.15 and ppm; and/or above 0.2 ppm.

In one version of the medical insufflation device, the ozone generator includes an electro-ionic device in communication with the medical gas and configured to generate the ozonated medical gas by converting the at least a portion of the oxygen in the medical gas into ozone. In such a version, the electro-ionic device may include an emitter and a collector plate. The emitter may include an ionizer needle emitter with free ends coated with at least one of zinc, iridium, and/or tantalum. The emitter may include an ionizer needle emitter with free ends coated with carbon nanotubes. The electro-ionic device may employ an operational voltage of 10 kV to 100 kV. The electro-ionic device may have an air gap of 2 cm. The electro-ionic device may employ an electric field strength of at least 5 kV/cm.

In one version of the medical insufflation device, the device further includes a vent attachable to the patient body and configured to vent the ozonated medical gas from the patient body.

The vent may include an ozone decomposition element. The ozone decomposition element may include an ozone sensor providing ozone measurements as the vented medical gas is released to the atmosphere. The ozone decomposition element may act to maintain the vented medical gas at an ozone concentration of below ppm when the targeted amount of ozone is above 0.05 ppm.

In one version of the medical insufflation device, the device may be configured to output the ozonated medical gas up to 6 L/min and up to 8 psi while maintaining a targeted ozone concentration between 0.5 ppm and 2.5 ppm.

In one version of the medical insufflation device, the device may be is configured to output the ozonated medical gas between 1.5 L/min and 4 L/min and between 5.5 psi and 7.5 psi, while maintaining a targeted ozone concentration between ppm and 2.5 ppm.

Other aspects of the present disclosure include a system for sanitizing an enclosed space. The system includes an electro-ionic device, an ozone sensor, and a controller. The electro-ionic device is configured to generate and output ozone into the enclosed space. The ozone sensor is in communication with the enclosed space and positioned away from the electro-ionic device. The controller is in communication with the electro-ionic device and the ozone sensor and configured to control the amount of ozone generated and output into the enclosed space by the electro-ionic device based on the amount of ozone detected by the ozone sensor.

In a first version of the system, the enclosed space is an interior of an elevator car and the electro-ionic device is configured to generate and output ozone into the interior. The electro-ionic device is configured to operate in at least a first mode where the concentration of ozone is maintained at a predetermined level and a second mode where the ozone is generated at a concentration higher than the first mode.

In the first version of the system, the predetermined level of the first mode may be an ozone concentration of between 0.05 to 0.15 ppm. In the second mode, the controller may cause the electro-ionic device to generate ozone at the maximum capability of the electro-ionic device.

In the first version of the system, the system operates in the first mode or the second mode according to a present occupancy condition or an anticipated occupancy condition of the elevator car. The ozone concentration for the second mode may be any of the following, depending on a user selected or manufacture set setting: below 0.1 ppm; between 0.1 and 0.15 ppm; between 0.15 and 0.2 ppm; and/or above ppm. In such a case, depending on a user selected or manufacture set setting, the ozone concentration for the first mode may be below 0.05 ppm.

In a second version of the system, the enclosed space is an interior of a refrigerated space.

In the second version of the system, the interior of the refrigerated space includes a refrigerator, a freezer, a meat locker, a wine closet, a fur storage facility, a medical supply facility, or any other type of refrigerated space.

In the second version of the system, the system attempts to maintain the ozone concentration within the refrigerated space between 0.05 to 0.15 ppm. Alternatively, the system attempts to maintain the ozone concentration within the refrigerated space less than 0.05 ppm. In either case, the system may be configured to achieve either concentration range by being set at either, by user or factory setting, or cycled between either range by the controller.

In the second version of the system, the electro-ionic device includes an emitter and a collector plate. The emitter may include an ionizer needle emitter with free ends coated with at least one of zinc, iridium, and/or tantalum. The emitter may include an ionizer needle emitter with free ends coated with carbon nanotubes.

In the second version of the system, the electro-ionic device may employ an operational voltage of 10 kV to 100 kV. The electro-ionic device may have an air gap of 2 cm. The electro-ionic device may employ an electric field strength of at least 5 kV/cm.

Yet other aspects of the present disclosure include an HVAC system comprising a metal ductwork, an ozone generator at least a portion of which is located in the metal ductwork, a electro-ionic device at least a portion of which is located in the metal ductwork, an ozone sensor configure to detect levels of ozone in a space supplied by the ductwork, and a control system in communication with the ozone sensor and that controls operation of the ozone generator and the electro-ionic device.

In some versions of the HVAC system, electro-ionic device partitions air flow through the metal ductwork into multiple smaller partitions. The multiple smaller partitions may be in the form of multiple collector tubes arranged in a “honeycomb” or hexagonal arrangement. Each of the collector tubes may surround an emitter array assembly including a plurality of electrodes arranged axially along a central longitudinal axis inside a collector tube. The electrodes are may be shaped as at least one of a thin wire, a needle, a thin cut triangular sheet, a microneedle, a hair fiber, or a nanotube. The diameter of the collector tubes may range from 3 inches to 6 inches and the length of the electrodes may range from ¼ inch to ½ inch. All of the electrodes may have substantially the same length.

The collector tubes may be spaced from one another and the metal ductwork by being potted or secured in a conductive seal frame. The conductive seal frame electrically ties each of the collector tubes and the metal ductwork to a same reference voltage. The conductive seal frame blocks all passageways between the collector tubes and the metal ductwork, forcing air to flow between the electrodes and the collector tubes and through curved triangular gaps located between three internal collector tubes. Within each of these curved triangular gaps, ozone generating electrodes of the ozone generator are located to receive a large negative voltage and ionize the air along with generating ozone.

Positive voltage outputs of the electro-ionic device and the ozone generator are tied to the collector tubes, the metal ductwork, and an earth ground. A negative output from the electro-ionic device is connected to each of the emitter array assemblies. An operating voltage of the emitter array may be between 0.5 kV to 10 kV, and an operating voltage of the ozone generator may be between 5 kV and 30 kV.

The ductwork may be in the form of a rectangular cross section or round or rounded cross section. The ductwork may supply at least one of a living space or a non-living space including at least one of a space internal to a wall, floor or ceiling or an interstitial space. The air supplied by the HVAC system may be sent by selection of the control system to alternatively supply the living space or the non-living space. The control system may operate the ozone generator to operate at a purge rate and a conditioned rate, the purge rate being at much higher ozone levels than the conditioned rate. For example, the conditioned rate may be at 0.05 ppm or less.

The HVAC system may further include an ozone decomposition device in fluid communication with the ductwork, the decomposition device being used to reduce the amount of ozone present by decomposing it to diatomic oxygen (02). The ozone decomposition device may decompose ozone through at least one of the following decomposition mechanisms: adsorptive decomposition, catalytic decomposition, or photocatalytic decomposition. The absorptive decomposition may employ an activated charcoal filter. The catalytic decomposition may employ a filter comprising metal oxides of at least Mn, Co, Fe, Ni, Zn, Ag, Cu, Pt, Pd, Rh, or Ce.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a diagrammatic view of an electro-ionic device in an HVAC system according to an exemplary embodiment of the present disclosure.

FIG. 2 is a diagrammatic view of the electro-ionic device of FIG. 1.

FIG. 3 is a front view of an emitter.

FIG. 4 is a front view of a collector.

FIG. 5 is an inflow side view of the electro-ionic device on the left, and an outflow side view of the electro-ionic device on the right.

FIG. 6 is a cross-sectional view of the electro-ionic device replacing a section of a duct.

FIG. 7 is an isometric view of an embodiment of the electro-ionic device positioned within a duct.

FIG. 8 is a side view of the embodiment of the electro-ionic device of FIG. 7 positioned within a duct and in a non-deployed state.

FIG. 9 is a side view of the embodiment of the electro-ionic device of FIG. 7 positioned within a duct and in a deployed state.

FIGS. 10A and 10B are, respectively, side views of an embodiment of an electro-ionic device positioned within a duct in a non-deployed state, and a deployed state.

FIGS. 11A and 11B are, respectively, side views of an embodiment of an electro-ionic device positioned within a duct in a non-deployed state, and a deployed state.

FIGS. 12A and 12B are, respectively, side views of an embodiment of an electro-ionic device positioned within a duct in a non-deployed state, and a deployed state.

FIGS. 13A and 13B are, respectively, a top view and a front, right-side, top perspective view of an embodiment of an ozone decomposition device.

FIGS. 14A and 14B are, respectively, a back, left-side, bottom perspective view and a front, right-side, top perspective view of an embodiment of a boot register positioned within subfloor of a building.

FIG. 15 is a schematic view of an HVAC system integrated with an ozone system and an electro-ionic device.

FIG. 16 is a schematic of a suitable computing and networking environment that may be used to implement various aspects of the present disclosure.

FIG. 17 is an isometric view of an embodiment of the electro-ionic device positioned within a rectangular duct.

FIG. 18 is an isometric view of an embodiment of the electro-ionic device positioned within a cylindrical duct.

FIGS. 19A and 19B are, respectively, side views of an embodiment of an electro-ionic device positioned onto and inside an elevator car.

FIG. 20 is a schematic of a suitable controller environment that may be used to implement various aspects of the present disclosure.

FIG. 21 is a front view of an embodiment of an electro-ionic device positioned inside a refrigerator.

FIG. 22 is a schematic view of an exemplary insufflation system including an electro-ionic device for generating ozone.

FIG. 23 is a schematic view of another exemplary insufflation system including an electro-ionic device for generating ozone.

### DETAILED DESCRIPTION

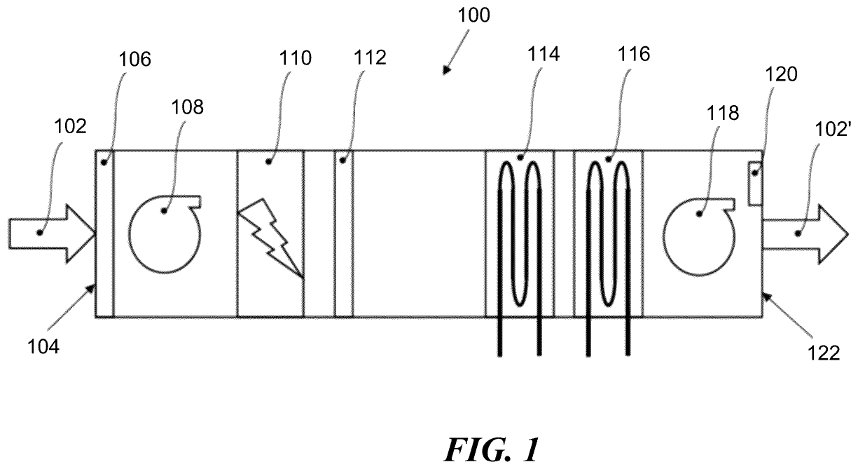

In an embodiment, as seen in the diagrammatic view of FIG. 1, an electro-ionic device 110 may be incorporated into an HVAC system 100 to mitigate the environmental loading of biopathogens in a given space. A system of this nature may be helpful to mitigate the spread of the COVID-19 virus, among other viruses, bacteria, and fungal particles, and allow for reinstatement of economic systems and return to work with lower risk of infection. The ionization technology described herein can mitigate the external environment in closed spaces such as, for example, office buildings, auditoriums, airplanes, and locations where people congregate to work or interact socially.

In the current understanding of airborne transmission of COVID-19, when people exhale, speak, or cough, they extrude droplets of saliva and mucous, many of which may contain virus particles. These droplets are usually large and can be well mitigated by existing filtration technology. It is over time that these larger droplets evaporate and become smaller, yet still carry viral particles. Some of these droplets may become embedded in masks or filters and then subsequently dislodge to the outside environment or into the lungs. These smaller droplets in the submicron size-range may still carry viable virus into ventilation systems, which have numerous metallic surfaces. It has been found that viruses, such as COVID-19, can thrive for an excess of three days on metallic surfaces. Such particles may become embedded within the HVAC filters and radiators or even trapped on the walls of the ventilation system. It is at least in this area that the electrostatic precipitator is helpful in mitigating the ventilation system from becoming a viral or biopathogen reservoir.

As seen in FIG. 1, the HVAC system 100 may include an inlet fan 108 that circulates inlet air 102 taking it from an intake vent 104 and moving it through the system 100 and out of an output vent 122 as outlet air 102′. The inlet air 102 may encounter a heating coil 114 if heating is required and/or an expansion coil 116 for air-conditioning or dehumidification if such is required. The system 100 may be controlled by a feedback controller, such as a thermostat with on/off timing capability. The thermostat is usually located in a living space and accessible to be manually set. The HVAC systems 100 may be controlled remotely through a wireless interface, such as Wi-Fi or Bluetooth through the Internet. Various parameters of the system may be controlled such as, for example, fan speed, temperature, humidity, and ozone level, etc. as a function of time.

The HVAC system 100 may include one or more fans such as an inlet fan 108 near the inlet 104 and optionally an outlet fan 118 near the outlet 122. The electro-ionic device 110 may be positioned upstream or downstream from the inlet fan 108. The electro-ionic device 110 may be activated from the same circuit as the inlet fan 108. In this instance, the electro-ionic device 110 will not activate unless the inlet fan 108 is activated; therefore, the unit will be powered by the same power supply that powers the inlet fan 108 that drives the air through the HVAC system 100. In another instance, the electro-ionic device 110 may be continually operated without regard to the state of the inlet fan 108. The electro-ionic device 110 may be AC powered or DC powered depending on the particular application.

In certain instances, the electro-ionic device 110 may be retrofitted to existing HVAC systems 100 so the heating coil 114 and the cooling expansion coil 116 remain in place and older flow controls remain in place. Conventional HVAC systems often require an upgrade when additional filter media is introduced into the system. For instance, the additional filter media may put a strain on the function of the circulating fan, which leads to earlier burnout of the fan and/or preemptive replacement of the fan. The fans also consume more energy under this type of strain. As opposed to adding additional filter material as a means to filter unwanted particles from the air system, adding the electro-ionic device 110 described herein into an existing HVAC system 100 has the advantage of minimal strain on the existing components of the system 100.

In certain instances, the HVAC system 100 may be built as-new with electro-ionic device 110 part of the overall system 100. In some instances, as seen in FIG. 1, the HVAC system 100 may also have one or more HEPA filters 106, 112 and the electro-ionic device 110 can be located proximally upwind to at least one of the HEPA filter 112, so that the HEPA filter 112 catches particles which are now subjected to higher levels of ozone to help ensure that there are no viral life particles within the HEPA filter. Effectively, the downstream HEPA filter 112 is sanitized in real time and is unlikely to become a viral reservoir. An ozone sensor 120 may be located just at the outlet vent 122 as this will help with control of the ozone levels within the system 100 itself.

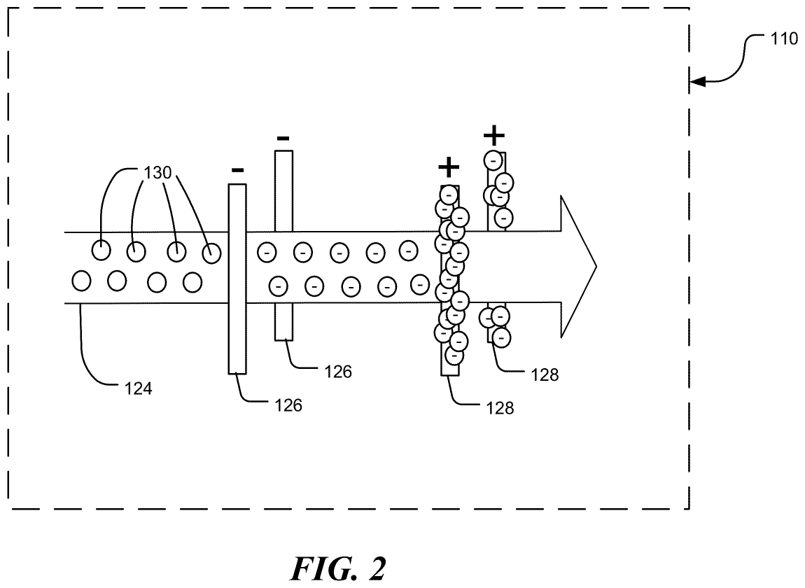

As seen in the FIG. 2, which is a diagrammatic view of an electro-ionic device 110 with a flow of air 124, the electro-ionic device 110 includes one or more emitters (negative conductor) 126, and one or more collectors (positive conductor) 128. As seen in the figure, there are two emitter 126 posts that are charged with a negative voltage as indicated by the negative sign above each post, and there are two collector 128 posts that are charged positively as indicated by the positive sign above each post. The flow of air 124 generally is representative of fluid flow through a duct of an HVAC system. Within the flow of air 124 are particles 130 such as dust, viral particles, bacterial particles, fungal particles, or the like. The flow of air 124 is left-to-right in FIG. 2. Prior to passing through the emitters 126, the particles 130 are relatively chargeless. Upon passing the emitters 126, the particles 130 pick up a negative charge because of the high negative voltage of the emitters 126, as indicated by the negative sign within the particles 130. Downstream of the emitters 126 are the collectors 128. The negatively charged particles 130 are moving with the flow of air 124 towards and through the collectors 128, which have a high positive charge. The particle 124 are attracted to the positive charge of the collectors 128 and attach themselves on the collectors 128. The air 124 continues to travel past and through the collectors 128 with many of the air particles with foreign matter (e.g., virus particles, bacterial particles, dust, fungus) having been attached to the collectors 128.

In certain instances, the emitters 126 and collectors 128 may be shaped and sized to fit with the ducts of the HVAC system 100 in a way that maximizes ionization of the airflow. The emitters 126 may be formed of stainless steel, or alloys containing nickel, chromium, manganese, combinations thereof, or another oxidation resistant conductive material. The emitter 126 may include various metal foils and/or coats with one or more of the previously mentioned alloys. The emitters 126 may be machined or laser cut into a series of rungs or posts. A portion of the rungs may be coated to help decrease the electron workforce and to improve the efficiency of the electro-ionic device 110. Such coatings may include manganese, iridium, tantalum, and zinc, among others. Reducing the electron workforce may permit a reduction in the emitter voltage and thereby improve the viability of the underlying power source as well as the underlying components.

In operation, a voltage potential is applied between the emitter 126 and the collector plates 128. In certain instances, the voltage potential is −10,000 volts to about −20,000 volts for the emitter 126. With the collector plates 128 being positively charged, this creates an electrostatic precipitator. When the emitter 126 is charged with respect to the collector plates 128, electrons build up on the electrodes of the emitter 126 at their respective tips. Depending on a number of factors, some electrons are transmitted across the gap between the emitter 126 and the collector plates 128. Preferentially, electrons attach to small airborne particles flowing through the duct of the HVAC system and, in particular, though the gap between the emitter 126 and collector plates 128, thereby imparting a negative charge thereto. These charged particles can be precipitated out and/or attracted to the nearby positively charged collector plates 128 creating an inertial diversion. The energizing voltage may be DC or pulsed with various frequencies.

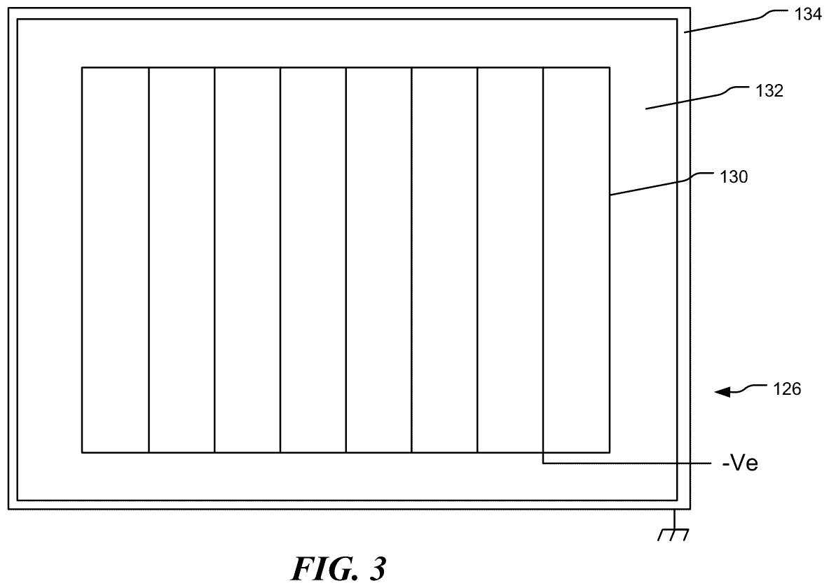

In certain instances, as seen in FIG. 3, which is a front view of an embodiment of the emitter 126 of the electro-ionic device 110, the emitter 126 may include a wire grid or mesh (e.g., conductive mesh) 130 in the form of a series of vertically oriented wires with a rectangular wire frame that encloses the series of vertically oriented wires that are spaced apart from each other so as to permit airflow between the wires. An insulating frame 132 encloses the wire grid 130 on four sides. The frame 132 is enclosed in an outer frame otherwise referred to as a duct frame 134 that extends around the insulating frame 132 on all four sides. The duct frame 134 is sized to fit snugly within the interior space of the duct of the HVAC system. The wire grid 130 is coupled to a voltage source that is configured to supply between about −10,000 volts to about −20,000 volts. The duct frame 134 is grounded to the surrounding ductwork to which it is secured therein.

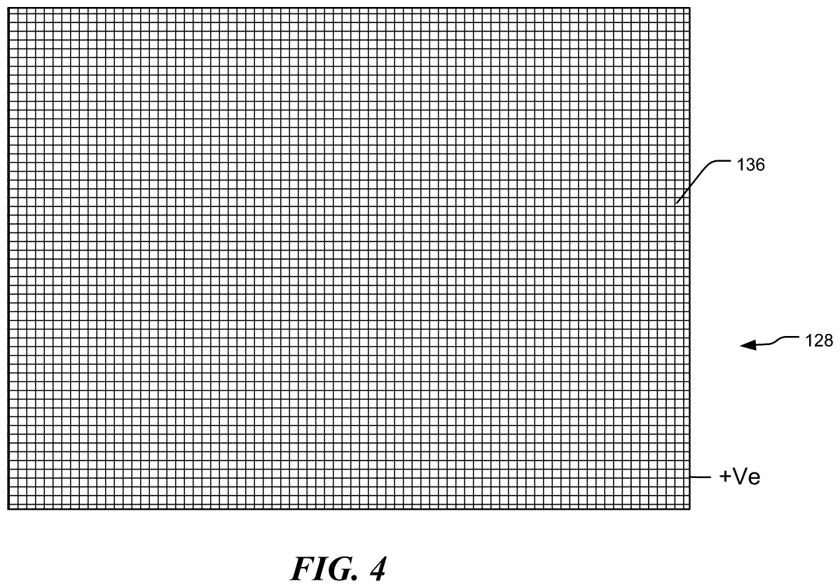

FIG. 4 depicts a front view of an embodiment of a collector 128. The collector 128 may include a wire grid 136 in a rectangular shape. In certain instances, a positive voltage charge may be applied to the collector 128 so as to form an electrostatic precipitator. The wire grid 136 is sized to fit within the duct frame 134 shown in FIG. 3. That is, the duct frame 134 is sized to either fit within the existing ductwork or replace a section of ductwork. In the case of replacing a section of ductwork, the existing ductwork could be cut and removed and replaced with the corresponding size of duct frame 134. The duct frame 134 houses the emitter 126 of FIG. 3 and the collector 128 of FIG. 4. In this say, both the emitter 126 and collector 128 span substantially the entire cross-section of the existing ductwork to treat substantially all of the passing airflow.

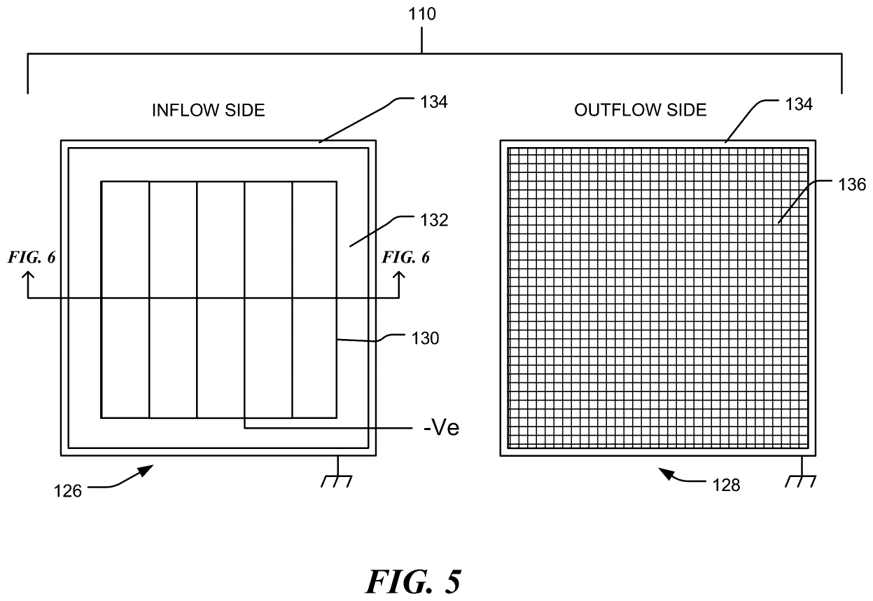

FIG. 5 shows an embodiment of the electro-ionic device 110 from an inflow side (left) and the outflow side (right). The inflow side depicts the emitter 126 housed within the duct frame 134, and the outflow side depicts the collector 128 housed within the same duct frame 134. The emitter 126 includes the wire grid 130, which is negatively charged, and the insulated frame 132 that encloses the wire grid 130. The duct frame 134 encloses the insulated frame 132. The collector 128 includes the closely spaced wire grid 136.

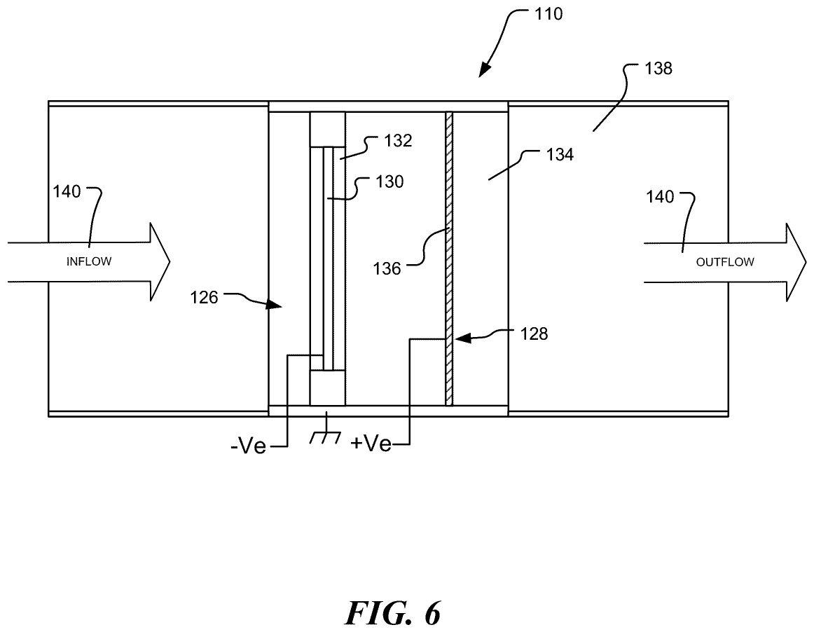

FIG. 6 depicts a cross-sectional view of the electro-ionic device 110 with the section taken at mid-height, as identified by the section-line in FIG. 5 on the left. As seen in FIG. 6, the duct frame 134 in the form of a four-sided frame replaces a section of the existing duct 138 of the same size. As seen in FIG. 6, the duct frame 134 is co-extensive with the existing duct 138. Housed within the duct frame 134 is the emitter 126 and the collector 128, both spanning across the entire duct. The emitter 126 includes the wire grid 130 that is housed within the insulating frame 132, which is fitted against the duct frame 134. The wire grid 130 is coupled to a negative voltage. The emitter 126 is upwind of the collector 128 such that the particles within the airflow 140 are negatively charged as it flows through the wire grid 130. The negatively charged particles in the air then flow through the wire grid 136 of the collector 128 and the particles are attracted and collected on the wires of the collector 128. The air continues to flow through the system. In certain instances, the duct(s) of the HVAC system 100 may not include dedicated collector plates 128, but may instead rely at least partially on the ductwork to attract the negatively charged ions. In this way, the electro-ionic device 110 would function as an ion generator.

Control of the electro-ionic device 110 can be exerted both by voltage modulation between the emitter 126 and collector 128 of the electro-ionic device 110 or through duty cycle modulation. For example, at night during absence of the workforce in a given building, the HVAC system 100 may generate higher levels of ozone to significantly sterilize living organisms within the HVAC system itself. This could also be a significant safety mechanism if implemented in airplanes that often circulate air in crowded areas and are often associated with spread of airborne diseases. A secondary control of ozone levels may be implemented near the thermostat accessible living space and may function very much similar to the control applied to temperature. As discussed above, such ozone control can also be integrated with existing temperature and HVAC controls. This may be implemented in a separate ozone control or integrated into an established HVAC control. Ozone level in the air greater than 0.1 ppm may irritate the respiratory tract and may not be conducive to good health. For this reason, the system 100 may implement several ozone sensors to provide better feedback and control for the electro-ionic device 110. Because of the fine control that could be achieved in this manner, higher levels of ozone in the building space may be acceptable when there is absence of people therein. Under such conditions, ozone may disinfect all surfaces, including floors, walls, ceilings, desktops, countertops, etc.

As described above with respect to the electro-ionic device 100, a high voltage positioned in the small gap between the emitter 126 and collector 128 generates high levels of localized ozone by virtue of their discharge. By using this same high voltage in the range of 10 kV to 20 kV and introducing a larger gap between the emitter 126 and collector 128, such as between 1 to 4 inches, less ozone is produced, but a significant cloud of electrons is emitted. The electrons have a tendency to latch onto small submicron particles and impart a negative charge upon them. In some embodiments, the walls of the ventilation ducts are used as the collector due to their electrical conductivity resulting in a large collector surface area. Accordingly, the device described herein may be used to convert an existing HVAC into an extended electrostatic precipitator that retains small airborne particles, including biopathogens, and prevents their exit into an occupied room. The concurrent use of purging ozone will help ensure that the trapped biopathogens on the collector 128 will be sanitized and the rendered noninfectious. Although the walls of the ventilation ducts are described as an extended collector, any conductive material can be used as an extended collector.

The additional functionality of an electrostatic precipitator in combination with an ozone generator is described herein. In one embodiment, the electrostatic precipitator device has a width of approximately 5 inches, which lends itself well to position into an existing ventilation system. The device may be positioned in a duct system downwind from the heater and cooling coils and particularly, downwind from the ozone generator, which is located upwind from the heating cooling coils and the standard HVAC filter. There are at least two connections to this unit, one of which is the negative terminal of the high voltage part and is connected to the emitter thin steel wires that crisscross the lumen of the of the HVAC vent. Because of their negative voltage polarity, they have a tendency to emit electrons to a distance of 1 inch to about 3 inches. The collector grid 128 is referenced to electrical ground where the positive terminal of the voltage generator connects and is in continuity with the vent system. Located within the gap between the negatively charged thin wires of the emitter 126 and the collector grid 128 is the high voltage potential that facilitates the emission of electrons. When small particles pass through both grids, they acquire a negative charge. Some of these charged particles will collide with the collector grid 128 and others will pass through becoming attracted to the extensive conductive duct walls and diverted away from the air stream.

The ionizer devices described herein are designed to be easily inserted into the ducts of HVAC systems, in some instances across its shorter cross section. It is designed to accommodate several different duct size profiles by virtue of midpoint positioning adjustability and emitter voltage adjustability. To accommodate different capacity HVAC systems, basic capacity units may be provided for systems that serve spaces up to 10,000 square feet, such as 5,000 square feet spaces and large capacity units may be provided for systems that serve spaces over 10,000 square feet.

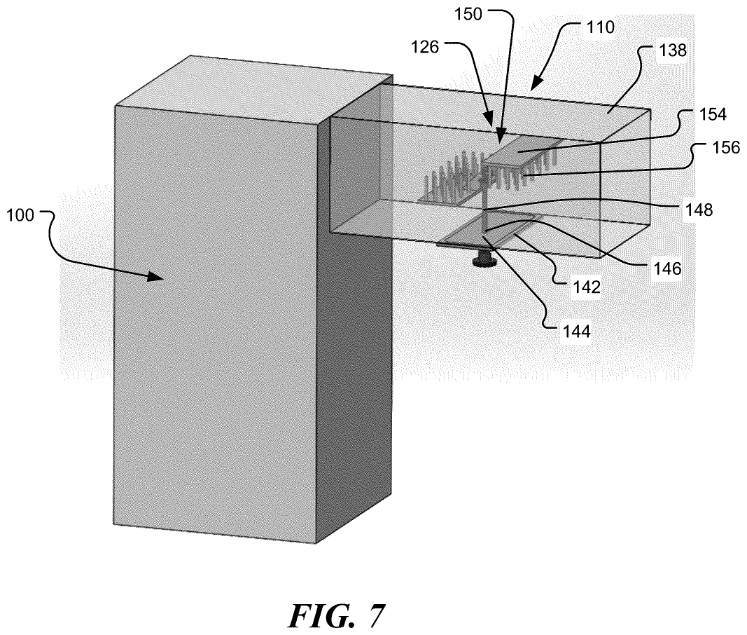

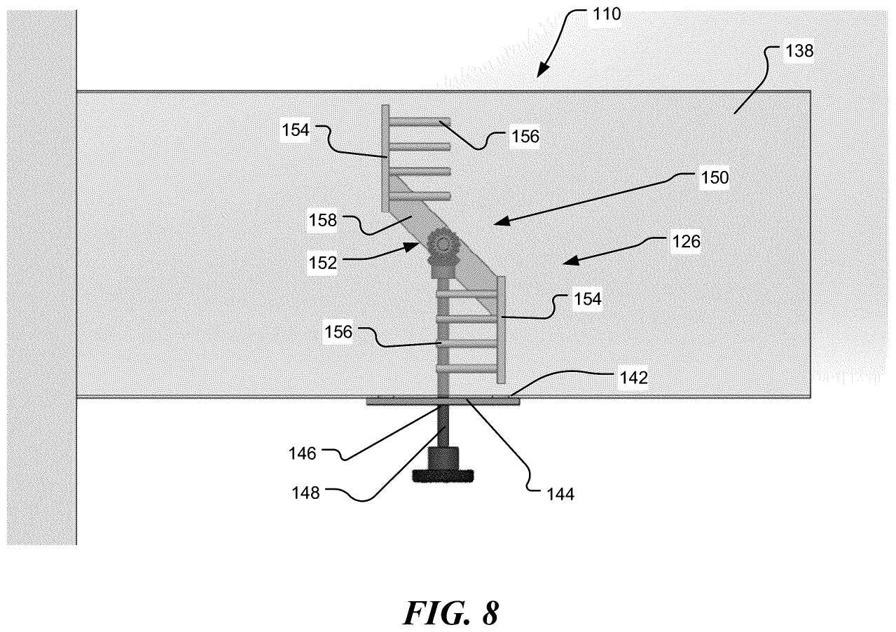

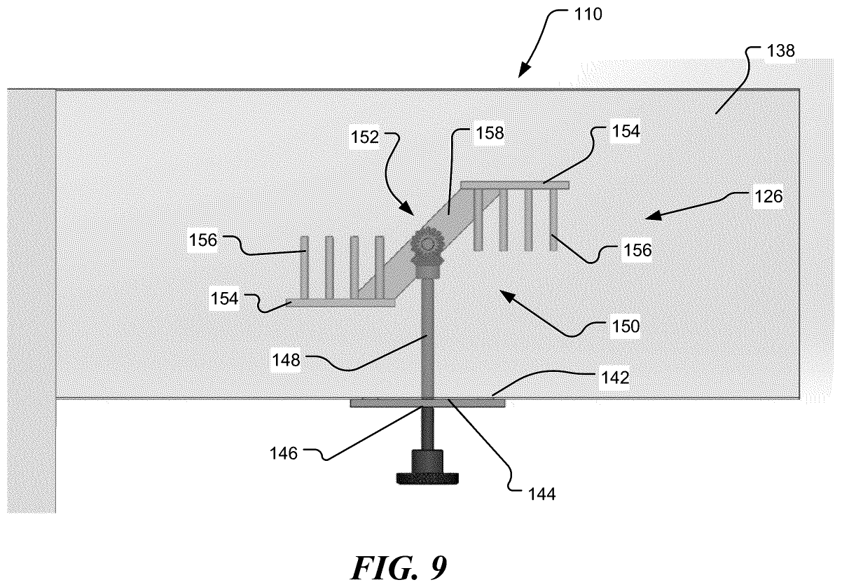

In an embodiment as shown in FIGS. 7-9, the electro-ionic device 110 may include an adjustable or deployable emitter 126 that is shown located in a mid-portion of the smaller cross section of a duct 138. FIGS. 7 and 9 show the emitter 126 in a deployed state, whereas FIG. 8 shows the emitter 126 in a non-deployed state. In the deployed state, shown in FIGS. 7 and 9, the emitters 126 are deployed horizontally after insertion into the duct 138. In the non-deployed state, shown in FIG. 8, the emitters 126 are vertical so as to permit insertion of the emitters 126 into an opening (e.g., square cutout) 142 in the duct 138. The deployable nature of the device 110 permits a relatively small opening 142 to be made in the duct 138.

As seen in FIGS. 7-9, the electro-ionic device 110 includes a base plate 144 sized to cover the opening 142 formed into a side of the duct 138. The base plate 144 includes a central opening 146 with an adjustable rod 148 extending there through. The rod 148 is adjustably coupled to an emitter assembly 150 at an end. The rod 148 can be adjusted in height relative to the base plate 144 so as to position the emitter assembly 150 at a particular height within the duct 138, such as at a mid-height within the duct 138. In the illustrated embodiment, the rod 148 is coupled to the emitter assembly 150 via a worm gear assembly 152 (as seen in FIGS. 8 and 9), whereby rotation of the rod 148 about a central axis causes the emitter assembly to transition between the non-deployed and deployed states, as shown in FIGS. 8 and 9, respectively.

The emitter assembly 150 includes a pair of parallel plates 154, each with conductive rods 156 coupled thereto. The plates 154 are rigidly coupled together via a support member 158 in the form of a bar, as seen in FIGS. 8 and 9. The emitter assembly 150 is arranged with the conductive rods 156 cantilevered off of their respective plates 154. The free ends of the rods 156 from one plate 154 opposes the free ends of the rods 156 from the other plate. As seen in FIG. 7, there is a series of rods 156 on the plate 154 that generally spans the surface of the plate 154. In this way, the rods 156 span the width of the duct 138. The rods 156 may be shaped as, for example, a thin wire, a needle, a thin cut triangular sheet, a microneedle, a hair fiber, or a nanotube.

In certain instances, the pair of plates 154 may be oriented coplanar with each other. In such an instance, the plates 154 would both be positionable at the midpoint of the duct 138. As seen in FIG. 9, the plate 154 on the left is closer to the lower duct 138, and the plate 154 on the right is closer to the upper duct 138. In order to minimize chances of a short circuit, the device 110 may be modified such that the pair of plates 154 are coplanar such that they both can be positioned at the midpoint of the duct 138 (i.e., equidistant between the upper and lower duct surfaces). In this orientation of coplanar plates 154, the rods 156 may extend in the same direction or opposite directions.

The electro-ionic device 110 may be inserted into the duct 138 in the non-deployed state, as shown in FIG. 8. In this state, the emitter assembly 150 is oriented vertically, with the plates 154 vertical and the rods 156 horizontal. The base plate 144 can be coupled to the duct 138. The rod 148 can be adjusted vertically to center the emitter assembly 150 within the duct 138. The rod 148 can also be adjusted, in this instance by rotation, to rotate the emitter assembly 150 into the deployed state, as shown in FIGS. 7 and 9. In the deployed state, the plates 154 are generally parallel with the upper and lower ducts, as well as with the direction of airflow through the duct 138. And the rods 156 are positioned perpendicular to the flow of air through the duct 138.

The rods 156 of the emitter assembly 150 will have a high voltage negative charge so that electrons emitted from the sharp ends are directed toward the grounded outer walls of the duct system, which are often made from conductive steel or aluminum. High voltage wires may be fitted through the rod 148 or otherwise through the base plate 144. The air space between emitter sharp pointed rods 156 and outer walls of the duct 138 then experiences a significant voltage gradient. As air flows through this voltage gradient space, the particles in the air flow become charge carriers and are thereby attracted and diverted to the duct walls 138, which now have the additional function of acting as an extended collector beyond their original function of containing and directing air flow. In essence, the activation of the electro-ionic device 110 within the duct system of the HVAC system 100 creates an extended electrostatic precipitator.

To further improve efficiency of the ionizer needle emitter, the ends of the rods 156 may be coated with other metals such as zinc, iridium, and/or tantalum to reduce electron emission work force and oxidative corrosion. Additionally or alternatively, the rods 156 may involve coating the sharp projections with carbon nanotubes to improve the efficacy of the emitter 126. Adherence methods for surface coating metals with carbon nanotubes have been optimized for electron emission. During manufacturing, carbon nanotubes may be oriented vertically by applying a strong vertical electric field during thermal, chemical, or plasma deposition.

The length of the rods or needles 156 may be in the range of 0.5 centimeters (cm) to 3 cm to accommodate most ducts 138. In certain instances, other lengths are possible. In any case, the rods 156 extend from the plates 154 by substantially the same length to help ensure a uniform electric field to which the flowing air is subjected. Experimentation revealed that 10 kV works well for particle reduction per 2 cm air gap or 5 kV/cm electric field strength (voltage divided by gap distance). In one embodiment, the device 110 uses 40 kV induction on the emitter 126 with 2 cm rods or needles 156 to cover airflow through a duct 138 whose smaller cross section is in the range of 20 cm (8 cm on each side plus 4 cm for needles, 8+8+2+2=20 cm). Because ducts 138 come in different cross sections, the emitter devices described herein are able to be used in different duct cross sections, and are further able to have midpoint positioning adjustability and inductive voltage variability. For most situations, the operational range of the voltage may be between 10 kV and 100 kV. Testing showed that with air gaps of 2 cm and a collector 128 length of 4 cm, >95% particle reduction was achieved with airflows of 30 liters/minute and as high as 80 l/m through the 2 cm gap and 10 kV field gap. Extended collector surface area of the duct system will produce improved particle reduction with higher airflows, a smaller emitter 126, and lower voltages.

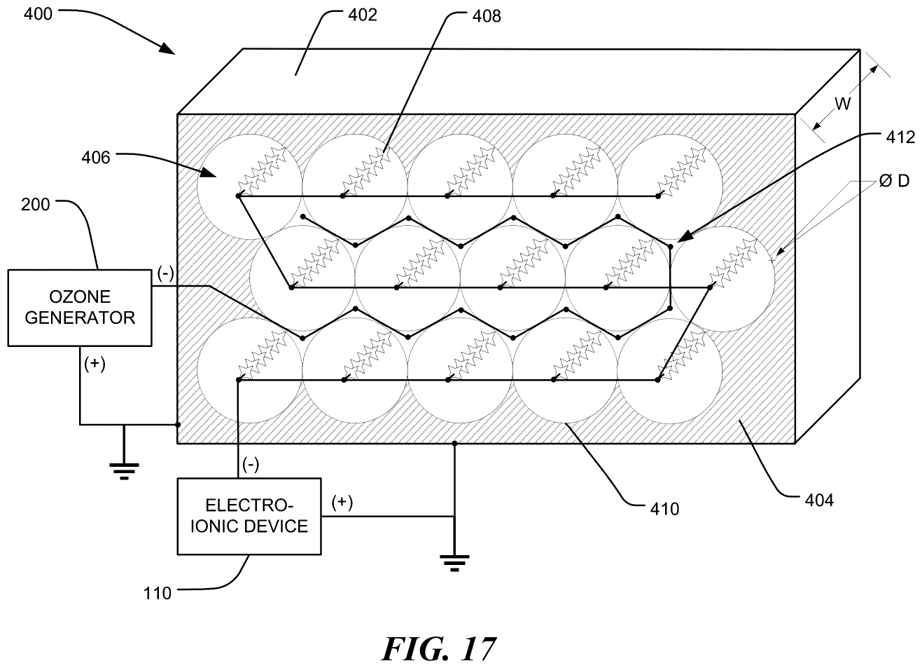

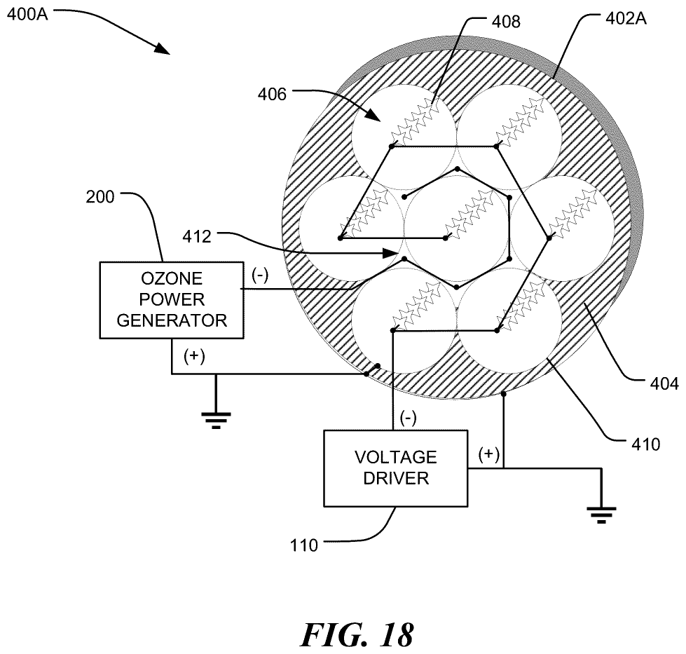

Generally, less ozone may be produced at a lower electric field strength, thus, it may be desirable in some instances to operate at a lower electric field strength for effective particle reduction and low ozone production. This may be achieved by minimizing the gap distance between the emitter 126 and collector 128. By doing so, the ability to produce high electric fields and, consequently, large amounts of ozone may be easier to achieve. For example, returning to the example above, where 10 kV was found to work well over a gap distance of 2 cm resulting in an electric field strength of 5 kV/cm, if the gap distance is reduced to 1 cm, then the voltage only needs to drop to 5 kV to obtain substantially the same level of particle reduction. At that lower voltage, less ozone may be produced. At the same reduced gap distance of 1 cm, an applied 10 kV potential may provide the same ozone production with improved particle elimination. Therefore, it may be desirable to reduce the gap distance between the emitter 126 and the collector 128. It was found that desired particle reduction may be achieved with an electric field strength of 1-5 kV/cm and desired ozone production may be produced with an electric field strength of 10-15 kV/cm. In addition, it may be considered safer to operate an electro-ionic device at lower voltages. Embodiments of electro-ionic devices that achieve these results are discussed in more detail with respect to FIGS. 17 and 18.

Activation of a purge cycle floods the living space with higher levels of supercharged oxygen (SO). Supercharged oxygen is biologically toxic or at least irritating at levels of 0.2 ppm/2 hours (OSHA standard) or 0.1 ppm/8 hours (OSHA standard for work environments). In a maintain mode of operation, it is intended that the living space level of ozone is maintained below the set level which usually will be below 0.1 ppm and the duct system at higher levels to maintain virucidal activity.

In the purge mode, the operator may set the target SO in the living space and its duration. When the living space is not ventilated with outside air, it was found that the average half-life of residual SO is of the order of 20 minutes. If more rapid degradation is desired, an enhanced external ventilation can be automatically implemented and or catalytic degradation device within the vent system and/or living space can be activated.

For the purge mode, the operator can set the initiation time and duration of purge as well as the maximum oxidant level within the living space to be maintained. A link to existing security system motion detectors may be used as third step in safety activation of our purge cycle with a given space in addition to activation controls.

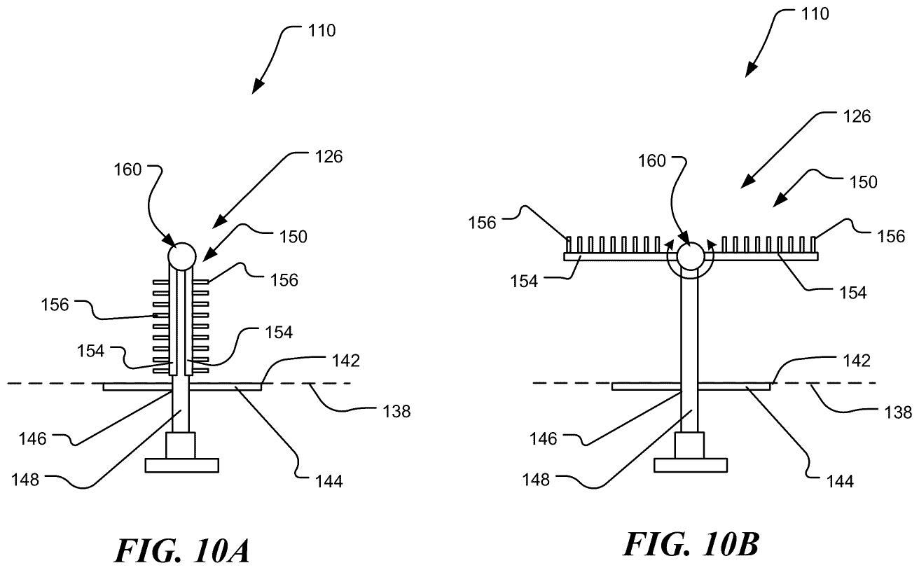

FIGS. 10A and 10B depict, respectively, an embodiment of an electro-ionic device 110 in a non-deployed state, and a deployed state. The electro-ionic device 110 is similar with respect to the device shown in FIGS. 7-9, except the device in FIGS. 10A and 10B includes a pair of plates 154 that are movable relative to each other and are rotatable about a central joint 160. In the non-deployed state of FIG. 10A, the plates 154 are parallel with each other and vertically oriented with the rods 156 extending horizontally in opposite directions. In this way, the width of the emitter assembly 150 is narrower than the base plate 144 and the opening 142 in the duct 138. Once the emitter assembly 150 is inserted into the duct 138, the rod 148 may be used to vertically position and secure in place the height of the emitter assembly 150. Then the rod may be used to deploy the emitter assembly 150 into the deployed state, shown in FIG. 10B. Deployment of the emitter assembly 150 may be similar to the deployment of an umbrella where the rod 156 is pulled relative to emitter assembly 150 and a linkage transitions the emitter assembly 150 from the non-deployed state to the deployed state. In the deployed state, the plates 154 are parallel with each other with the rods pointed vertically in the same direction as each other. It is noted that in a different embodiment, the rods 156 could face in opposite directions, or the rod 156 on both plates could face downward in the deployed state. The embodiment of the electro-ionic device 110 shown in FIGS. 10A and 10B may be utilized in pairs. That is an additional device 110 could be positioned upstream or downstream of the device 110 shown in the figures. The second device could be utilized on the opposing wall of the duct 138. Both devices 110 may be connected to the same voltage source, and the second device may be grounded to the surrounding duct 138.

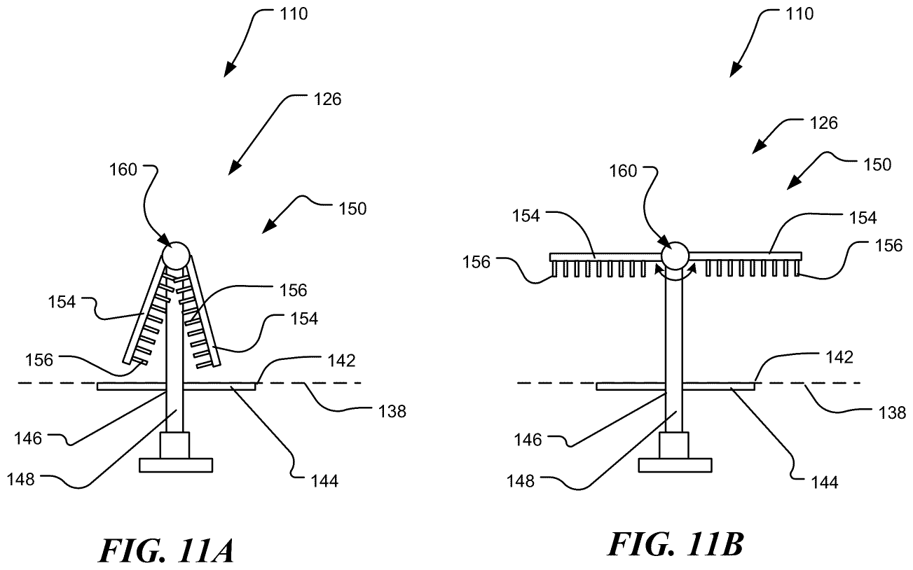

FIGS. 11A and 11B depict, respectively, an embodiment of an electro-ionic device 110 in a non-deployed state, and a deployed state. The electro-ionic device 110 is similar with respect to the device shown in FIGS. 10A and 10B, except the device in FIGS. 11A and 11B includes conductive rods 156 oriented on the opposite sides of the plates 154. In the non-deployed state of FIG. 11A, the plates 154 are angled downward and are rotated inward towards the rod 148. As seen in the figures, the width of the emitter assembly 150 is narrower than the base plate 144 and the opening 142 in the duct 138. Once the emitter assembly 150 is inserted into the duct 138, the rod 148 may be used to vertically position and secure in place the height of the emitter assembly 150. Then the rod may be used to deploy the emitter assembly 150 into the deployed state, shown in FIG. 11B. Deployment of the emitter assembly 150 may be similar to the deployment of an umbrella where the rod 156 is pulled relative to emitter assembly 150 and a linkage transitions the emitter assembly 150 from the non-deployed state to the deployed state. In the deployed state, the plates 154 are parallel, and coplanar with each other. The rods 156 are pointed vertically and positioned on the bottom side of the plates 154. It is noted that in a different embodiment, the rods 156 could face in opposite directions. The embodiment of the electro-ionic device 110 shown in FIGS. 11A and 11B may be utilized in pairs. That is an additional device 110 could be positioned upstream or downstream of the device 110 shown in the figures. The second device could be utilized on the opposing wall of the duct 138. Both devices 110 may be connected to the same voltage source, and the second device may be grounded to the surrounding duct 138.

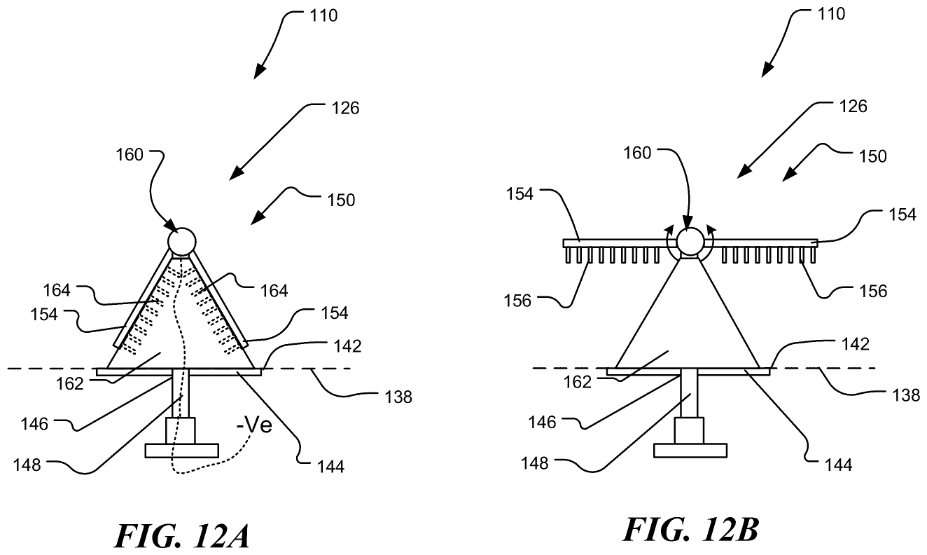

FIGS. 12A and 12B depict, respectively, an embodiment of an electro-ionic device 110 in a non-deployed state, and a deployed state. The electro-ionic device 110 is similar with respect to the device shown in FIGS. 11A and 11B, except the device in FIGS. 12A and 12B includes a base platform 162 positioned on the base plate 144 that includes recesses 164 for receiving the rods 156 when the device 110 is in the non-deployed state. As with the device in FIGS. 11A and 11B, there are a pair of plates 154 that are movable relative to each other and are rotatable about a central joint 160. In the non-deployed state of FIG. 12A, the plates 154 are positioned against the base platform 162, which is triangular in shape. In this state, the rods 156 are received within the recesses 164 of the platform 162, which may be constructed of a non-conductive material such as plastic. This protects the rods 156 when not in use (i.e., not in the deployed state). As seen in FIG. 12A, the width of the emitter assembly 150 is narrower than the base plate 144 and the opening 142 in the duct 138. Once the emitter assembly 150 is inserted into the duct 138, the rod 148 may be used to vertically position and secure in place the height of the emitter assembly 150. Then the rod 148 may be used to deploy the emitter assembly 150 into the deployed state, shown in FIG. 12B. Deployment of the emitter assembly 150 may be similar to the deployment of an umbrella where the rod 156 is pulled relative to emitter assembly 150 and a linkage transitions the emitter assembly 150 from the non-deployed state to the deployed state. In the deployed state, the plates 154 are parallel, and coplanar with each other with the rods 156 pointed vertically in the same direction as each other. In this embodiment, the rods are pointed downwards towards the base plate 144. It is noted that in a different embodiment, the rods 156 could face in opposite directions, or the rod 156 on both plates could face upwards in the deployed state, as in the device of FIGS. 10A and 10B. The embodiment of the electro-ionic device 110 shown in FIGS. 12A and 12B may be utilized in pairs. That is an additional device 110 could be positioned upstream or downstream of the device 110 shown in the figures. The second device could be utilized on the opposing wall of the duct 138. Both devices 110 may be connected to the same voltage source, and the second device may be grounded to the surrounding duct 138. FIG. 12A shows in dotted lines how the rods 156 can be electrically connected through the rod 148 to a voltage source. The other embodiments can be similarly connected through the rod 148 or otherwise.

In many of the embodiments described herein, the emitters 126 are positioned at a midpoint within the ducts 138 to avoid a short circuit. When deployed, the rods or needles 156 are in a vertical orientation, parallel to the sidewalls of the ducts 138. The rods or needles 156 are also oriented in a perpendicular direction at least one duct wall 138 that acts as a collector wall. With proper application of high-voltage this essentially becomes the configuration of an ionizer with a monopolar emitter 126 and collector configuration (duct wall). In certain instances, to fully cover the cross-section of the duct 138 using devices 110 with midpoint deployment of a monopolar device, a second device 110 can be deployed from the contralateral side and with the needle orientation being against the other wall.

FIG. 17 shows an exemplary embodiment of a vent interposed segment 400. The vent interposed segment 400 may include the ozone generator 200 and the electro-ionic device 110 in a relatively small vent segment having a width W. In some embodiments the width W may be between 2 inches and 8 inches to allow for easy installation into a new or existing ventilation system. The electro-ionic device 110 may achieve improved particle reduction by partitioning the air flow air into multiple smaller partitions, such as multiple collector tubes 410 arranged in a “honeycomb” or hexagonal arrangement. Each of the collector tubes 410 may surround an emitter array assembly 406 including a plurality of electrodes 408 arranged axially along central longitudinal axis inside each collector tube 410. The electrodes 408 may be shaped as, for example, a thin wire, a needle, a thin cut triangular sheet, a microneedle, a hair fiber, or a nanotube. In some embodiments, the diameter of the collector tubes 410 may range from 3 inches to 6 inches and the length of the electrodes 408 may range from ¼ inch to ½ inch. However, all installed electrodes 408 will have substantially the same length to help ensure a uniform electric field strength.

The emitter array assembly 406 and the collector tubes 410 may operate in similar manner as emitter 126/emitter assembly 150 and collector 128 described above, but may have an improved particle reduction due to having smaller gap distances between the electrodes 408 and collector tube 410. As discussed above, having a smaller gap distance may significantly lower the operating voltages, thereby not only improving the efficacy of the electro-ionic device 110, but also lowering the cost, complexity, and improving the safety of the driver electronics. In addition, the vent interposed segment 400 may achieve a more complete particle reduction as less air flow is able to bypass its functional ionization field. In some embodiments, the collector tubes 410 may be spaced from one another and the outer vent surface 402 by being potted or secured in a conductive seal frame 404, which may include a conductive epoxy or the like or a cutout in a conductive plate. The conductive seal frame 404 may electrically tie each of the collector tubes 410 and the outer vent surface 402 to a same reference voltage, such as earth ground. The conductive seal frame 404 may also block all passageways between the collector tubes 410 and the outer vent surface 402, forcing air to flow between the electrodes 408 and the collector tubes 410 and through curved triangular gaps located between three internal collector tubes 410. In each of these curved triangular gaps 412, the ozone generating electrodes may be positioned (see FIG. 17) to receive the large negative voltage and ionize the air along with generating ozone. Furthermore, the ozone generating electrodes may be bracketed with an electrically insulating material to maintain a fixed distance away from the collector tubes 410. In other embodiments, the curved triangular gaps 412 may be plugged to prevent air flow therein.

As shown in FIG. 17, the positive voltage outputs of the electro-ionic device 110 and the ozone generator 200 may be tied to the collector tubes 410, the outer vent surface 402, and earth ground. The negative output from the electro-ionic device 110 may be connected to each of the emitter array assemblies 406. Due to the relatively close distances of the electrodes 408 to the collectors 410, the operating voltage may range from 0.5 kV to 10 kV. The operating voltage of the ozone generator 200 may range between 5 kV and 30 kV. During testing, it was found that the energy consumptions for both the electro-ionic device 110 and the ozone generator 200 was less than 100 watts, and during some tests, it was found that the combined energy consumption was below 20 watts, less than 5 watts, and further less than 1 watt.

Although the vent interposed segment 400 includes both the ozone generator 200 and the electro-ionic device 110, each may be controlled by separate circuits and by different parameters. For example, the electro-ionic device 110 may be configured to operate only when the blower is on to reduce the energy consumption and prolong the longevity of the components, whereas the ozone generator 200 may be configured to operate based in part on a feedback system of one or more ozone sensors 120.

FIG. 18 shows another exemplary embodiment of a vent interposed segment 400A. The vent interposed segment 400A is substantially the same as the vent interposed segment 400 with minor differences in the geometry of the vent. For example, the vent interposed segment 400 as shown in FIG. 17 includes a rectangular outer vent surface 402, whereas the vent interposed segment 400A as shown in FIG. 18 includes a cylindrical outer vent surface 402A. Otherwise, these geometric differences, the two vent interposed segments 400 and 400A operate in substantially the same manner.

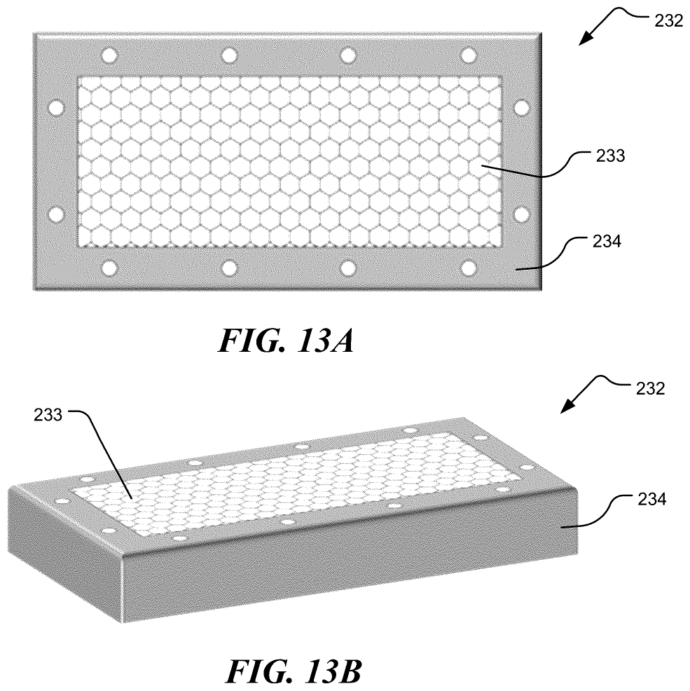

FIGS. 13A and 13B show an embodiment of an ozone decomposition device 232. The ozone decomposition device 232 may be used to reduce the amount of ozone present by decomposing it to diatomic oxygen (02). The ozone decomposition device 232 may decompose ozone through various decomposition mechanisms such as adsorptive decomposition (i.e., an activated carbon filter), catalytic decomposition (i.e., a filter comprising metal oxides of Mn, Co, Fe, Ni, Zn, Ag, Cu, Pt, Pd, Rh, and Ce), or photocatalytic decomposition. The ozone decomposition device 232 may include a porous filtrate grid 233, such as a honeycomb-shaped grid to allow airflow through or adjacent a high surface area of the decomposition filtrate. The filtrate grid 233 may be surrounded by a housing 234 to enable easy mounting to an existing boot register.

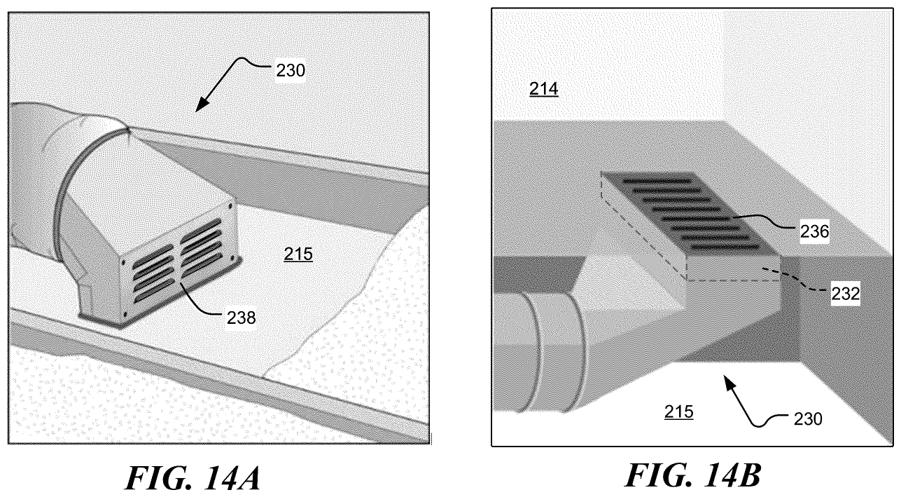

FIG. 14A illustrates an embodiment of a boot register 230 viewed from below a subfloor 215 of a living space 214, whereas FIG. 14B shows the boot register 230 from a split view above and below the subfloor 215. Although, the boot register 230 is shown installed within the subfloor 215 of a living space 214, it may alternatively be installed within the walls surrounding the living space 214. The boot register 230 may include an outwardly-opening ventilation register 236 which faces and opens into the living space 214 and an inwardly-opening ventilation register 238 which faces and opens into the subfloor or walls 215 behind the sheetrock. The outwardly- and inwardly-opening ventilation registers 236 and 238 may be actuated between an open and closed orientation via a mechanical linkage and slider or switch (not shown) positioned on the outside of the outwardly-opening ventilation register 236. Thus, the air flow may be directed to one, both, or neither of the living space 214 and the subfloor or walls 215. In other embodiments, the mechanical linkages may be opened and closed by a mechanical actuator (not shown) to remotely open or close the outwardly- and inwardly-opening ventilation registers 236 and 238. For example, it may be desirable to direct ozone into the subfloor or walls 215 for pest control, such as termite or other insect infestation or for mold and other bio pathogen mitigation. It also may be desirable to redirect some of the ozone into the subfloor or walls 215 instead of the living space 214 during an ozone purge, which is discussed in more detail below. As shown in FIG. 14B, the boot register 230 may optionally include an ozone decomposition device 232 positioned behind the outwardly-opening ventilation register 236.

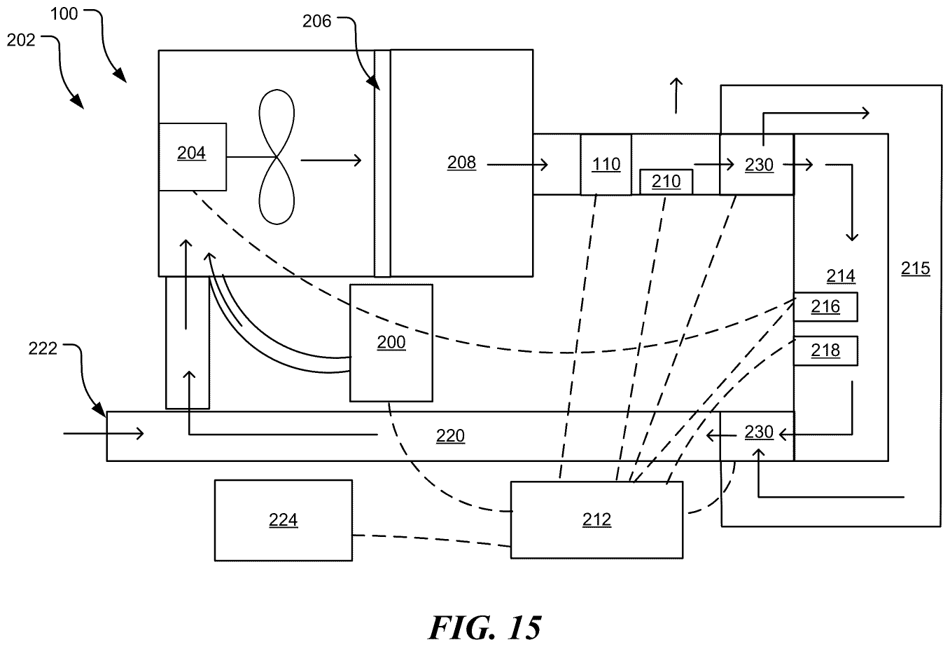

Another aspect of the present disclosure is an ozone generator 200 for use with an HVAC system 100, and is depicted in FIG. 15. The ozone generator 200 may be part of an ozone system 202 that further includes additional componentry to power, and control the delivery of ozone through the HVAC system 100. The ozone system 202 may be used with the electro-ionic device 110, or it may be a standalone unit. The ozone generator 200 may connect to an existing HVAC system 100 and functions to kill viruses, fungi, and bacteria in the ductwork and the space the HVAC system 100 is serving, while the electro-ionic device 110 primarily functions to capture the viral and bacterial particles and move them out of the airspace. When used together, the particles are captured, and killed.

The ozone generator 200 may generate ozone, inject it into the ductwork of the HVAC system 100, and circulate the ozone throughout the ductwork and room/building. The system 202 may monitor ozone levels in both the HVAC system and room/building via sensors and be able to control the generation of ozone based on feedback of ozone levels. The system 202 may be connected to a data logger or memory device, such as a computer, to allow for monitoring and logging of system parameters.

A schematic of the ozone system 202 is shown in FIG. 15. To begin, the ozone generator 200 is in fluid connection with the blower 204 so as to provide ozone to the blower 204 for circulating with existing air in the system 100 and fresh air. The blower 204 circulates the air and ozone mixture through a filter 206 and through a furnace or AC plenum 208. The air and ozone mixture goes through ducts and may pass through an electro-ionic device 110 as described previously. The electro-ionic device 110 is in communication with a controller 212. In certain instances, there is no electro-ionic device 110. Within the duct, is a supercharged oxygen (SO) sensor 210 that measures SO levels within the duct. The SO sensor 210 is in communication with the controller 212. This sensor 210 is located just prior to the living or working space 214. Also prior to the living space 214, the system may include at least one boot register 230 for directing air and ozone into the living space 214 and/or the subfloor and walls 215. As discussed above, the boot register 230 may include mechanical controls to open and close outwardly- and inwardly-opening ventilation registers 236 and 238 for directing air and ozone flow into the respective living space 214 and the subfloor or walls 215. As discussed above, the boot register 230 may include electromechanical control of the outwardly- and inwardly-opening ventilation registers 236 and 238 by the controller 212. Within the living space 214 is a thermostat 216 that is in communication with the blower 204 and the controller 212. There is also a second SO sensor 218 located in the living space 214 that is in communication with the controller 212. The living space 214 is in fluid communication with a return duct 220 via at least one return register. The return register may be similar to the boot register 230 to allow air from both the living space 214 and the subfloor or walls 215 to be directed into the return duct 220. In some embodiments, air and ozone from within the subfloor or walls 215 may be in fluid communication with the living space 214 via unintentional cracks, electrical outlets, etc. and may not require a return register having an opening into the subfloor or walls 215. The return duct 220 routes the conditioned air back to the blower 204 and is also met with a fresh air vent 222. The fresh air, conditioned air, and newly generated ozone are input into the blower for continued circulation. The ozone generator 200 is in communication with the controller 212. And the controller 212 is also in communication with a data logger 224. The arrows in FIG. 15 depict the direction of air and ozone flow through the system 202.

The controller 212 may include a computer and a panel mounted to the HVAC duct near the furnace, upstream from the HVAC filter. The controller 212 may include a user interface, such as a button that allows the user to change operation modes and a display screen or other visible indicators showing operation mode and ozone levels. The controller may control the ozone generator 200, blower 204, and the humidifier (not shown).

The various communications lines indicated by dotted lines in FIG. may be hard lines or wireless communications. There may be a primary wireless unit in the living space 214. This unit may be in communication with the ozone sensor 218 and may communicate with the controller 212 and/or other components. It may contain a display screen or other visible indicators showing operation mode and ozone levels and an audio alarm to alert occupants to high ozone levels. It may contain a key for activating the various modes of operation described subsequently.

The system may be able to operate in three exemplary operation modes: off; maintenance; and purge. In the Off mode, the system 202 will not generate ozone or interact with the HVAC system 100 but will continue monitoring ozone levels. In Maintenance mode, the system 202 produces safe levels of ozone when people are present in the living space. The system will maintain a user set ozone level, such as not to exceed 0.1 ppm average over 24 hrs. It should be recognized that other levels of ozone greater than or less than 0.1 ppm of ozone may be set, such as, for example or 0.05 ppm. Purge mode can produce high levels of ozone intended to disinfect surfaces and is not intended to be used when people are present. Purge mode may be utilized when the air and surfaces in the living space are desired to be sanitized. Using the ozone decomposition devices 232 may permit even higher levels of ozone to be produced with the HVAC system 100 than otherwise. Also, the controller 212 may partially or fully open or close the outwardly- and inwardly-opening ventilation registers 236 and 238 to help maintain a lower ozone concentration in the living space 214, during, for example, Maintenance mode. This control may be done based in part on the ozone level detected from the SO sensors 210 and 218. It may also be desirable to purge various zones or portions of the living space 214.

Supercharged oxygen is an encompassing term that describes oxidative injury to COVID19 viral particles. It encompasses O₃, —OH, H₂O₂ molecules which are generated concurrently when oxygen and water are subjected to UV light, plasma emission, high voltage gradient and variety of energy sources that can move and dislodge electrons from their usual low energy state. Differentiating peroxide gas, from ozone, from hydroxide is a matter of nomenclature and often used to circumvent regulatory constraints because any molecule that can kill a virus can be irritating and toxic at higher concentrations to living tissue such as lung if inhaled. Oxidizing surface proteins by virtue of dislodged electrons originating in the oxygen molecule whether the oxygen originates as O₂ or as H₂O is the underlying mechanism for the desired virucidal effect being implemented herein within the HVAC system. The system described herein may also activate existing HVAC humidifier function to increase water vapor presence which improves virucidal efficacy. With water a greater fraction of supercharged oxygen is in the form of gas hydrogen peroxide and as such exhibits less irritation to respiratory system than equivalent ozone levels in the absence of water vapor.

By combining this ionizer 110 with the ozone generator 200, along with a feedback control circuit, a significant particle reduction and neutralization is possible for existing circulating biologicals that potentially can cause harm by virtue of them being airborne bio pathogens. The technology herein accomplishes what prior devices have struggled to is not only particle enhance particle reduction but also virucidal and bio pathogen reductions in real time without significant modification of existing HVAC systems.

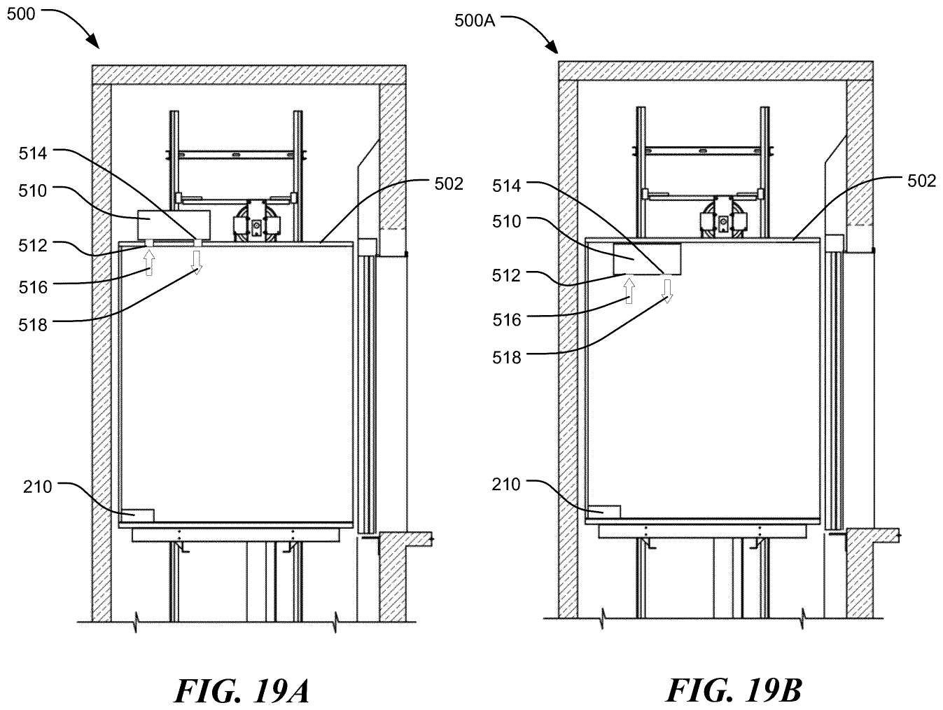

FIGS. 19A and 19B show exemplary embodiments of elevator sanitizing systems 500 and 500A, respectively. The elevator sanitizing systems 500, 500A may operate in a similar manner to the HVAC system 100 or the ozone system 202 but may be configured for an individual room or enclosed area, such as an elevator car 502. As shown in FIG. 19A, the electro-ionic device 510 may be positioned outside the elevator car 502 above the ceiling of the elevator car 502. The electro-ionic device 510 may be incorporated into an elevator car 502 during installation of the elevator car 502 or it may be retrofitted into an existing elevator car 502. The electro-ionic device 510 may be substantially the same as the electro-ionic device 110 or ozone generator 200 in that it includes the same subcomponents described above such as the emitter 126 and the collector 128 (both of the electro-ionic device 110 and the ozone generator 200 are capable of particle reduction and ozone production depending on the spacing between the emitter 126 and the collector 128 and the applied voltage).

Returning to FIG. 19A, the elevator car 502 may have a pair of openings that open into an inlet 512 and an outlet 514 of the electro-ionic device 510. The inlet may have a fan (not shown) configured to direct air 512 from inside the elevator car 502, through the electro-ionic device 510, and then back out into the elevator car with the generated ozone 518. In other embodiments not shown, the elevator car 502 may have its own dedicated ventilation system, in which case the inlet 512 and the outlet 514 may be in-line with such ventilation system, to direct air through the electro-ionic device 510 in a similar manner as described above. The elevator sanitizing systems 500 may also include the ozone sensor 210 positioned inside the elevator car 502 and configured to permit feedback control of the ozone emission in a similar manner as described above with respect to the ozone system 202. As shown in FIGS. 19A and 19B, the ozone sensor 210 may be located near the floor inside the elevator car 502. However, in other embodiments not shown, the ozone sensor 210 may be positioned in or near the inlet 512, the outlet 514, or anywhere else within the elevator car 502.

FIG. 19B shows the elevator sanitizing system 500A, which is substantially the same as the elevator sanitizing system 500 except for the electro-ionic device 510 being positioned inside the elevator car 502 against the ceiling instead of above it on the opposite side. Aside from the different placement of the electro-ionic device 510 with respect to the elevator car 502, the elevator sanitizing system 500A is substantially the same as the elevator sanitizing system 500.

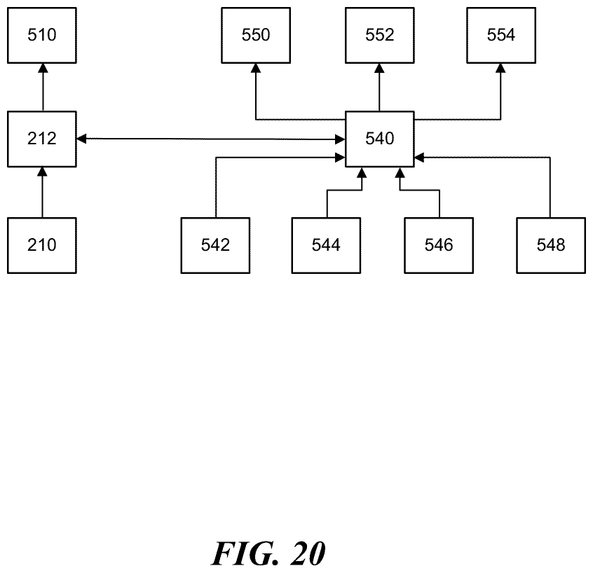

FIG. 20 shows an exemplary schematic regarding the sensing and control mechanisms of the elevator sanitizing systems 500 and 500A. The electro-ionic device 510 may be controlled by the controller 212 as described above with respect to the ozone system 202. For example, the controller 212 may control the electro-ionic device 510 to generate ozone in a first mode at a continuous low level steady state concentration or in a second mode a high level concentration for purging the elevator car 502. The ozone sensor 210 is configured to measure the level of ozone in the elevator car 502 and send a signal representative of this measurement to the controller 212. During the first mode, the controller 212 may control the electro-ionic device 510 to maintain a predetermined low concentration of ozone, such as a concentration between 0.05 to 0.15 ppm. During the second mode, the controller 212 may control the electro-ionic device 510 to output a maximum concentration of ozone that the electro-ionic device 510 is capable of generating, with the maximum concentration being higher than the predetermined low concentration.

To determine whether the controller 212 is operating in the first or second mode, the controller may receive a signal from an elevator controller 540. FIG. 20 depicts the controller 212 as a separate unit from the elevator controller 540, but in other embodiments, the elevator controller 540 may be configured to perform the functions of the controller 212. The elevator controller 540 may be connected to a position sensor 542. The position sensor 542 may comprise one or more sensors that output information relating to the position of the elevator car 502 within the building. Such sensors may include accelerometers, a cable encoder, among others. The elevator controller 540 may also include a door state sensor 544 that indicates whether the door is open or closed. In addition, the elevator controller 540 may receive information from one or two elevator dispatch sensors 546 for each floor of the building. These sensors include the up or down buttons that a user may press to summons the elevator car 502. Similarly, the elevator controller 540 may receive information from a number of destination sensors 548 corresponding to each floor in the building. These sensors specifically include the numerical button panel inside the elevator car 502 which a user selects for a given destination.

The elevator controller 540 may also control a number of functions based at least on the output of the sensors 542, 544, and 546. For example, the elevator controller 540 may control at least the movement 550 of the elevator car 502, the opening and closing 552 of the door, and the turning on and off 554 the elevator car ventilation system. Thus, the elevator controller 540 may send a signal to the controller 212 to begin or stop an ozone purge based on whether elevator car 502 has been emptied of passengers or is summoned to pick them up. For example, after the elevator controller 540 determines that no one is on the elevator, it may send a signal to begin an ozone purge and then take the particular elevator car 502 off-line for a predetermined time, such as 30 seconds. In some embodiments, the elevator controller 540 may send the instructions to begin an ozone purge until interrupted by a dispatch signal 546, at which point, it may communicate with the controller 212 and/or the ozone sensor 210 to cease opening the doors of the elevator car 502 until the ozone levels inside are at or below a predetermined safe concentration. The elevator controller 540 may also control the ventilation system to replace the air inside the elevator car 502 with new to expedite the lowering of the ozone to the safe concentration.

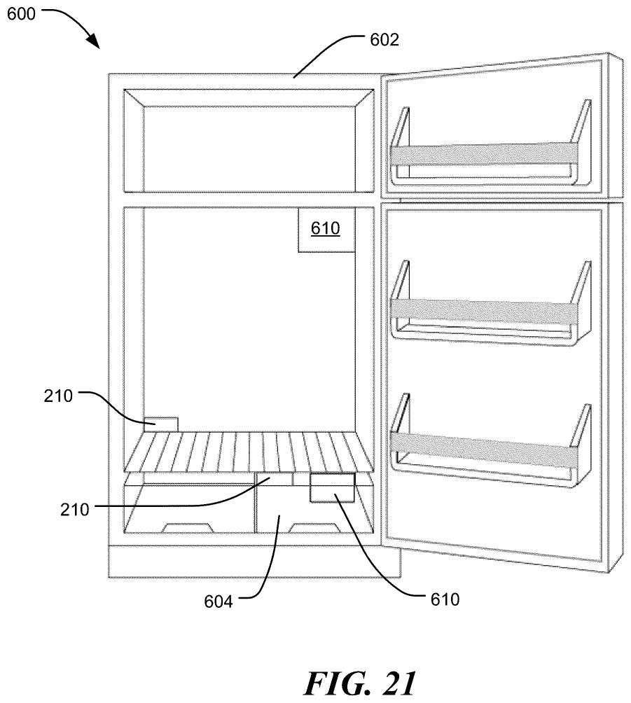

FIG. 21 illustrates an exemplary embodiment of a refrigerator sanitation system 600. It may be desirable to emit and maintain low levels of ozone inside a refrigerator 602 to inhibit the growth of or lower the growth rates of bacteria and fungi. Ozone may be particularly useful for inhibiting Listeria monocytogenes bacteria which tend to thrive at cold temperatures and anerobic microorganisms commonly found in refrigerators. In addition, it may be desirable to neutralize potential aldehyde groups associated with foul smells commonly found inside refrigerated food storage units. In addition to mitigating the smells found in a running refrigerator currently storing food, ozone may also mitigate the smell found in a closed door unplugged refrigerator. Overall, low level ozone treatment combined with refrigeration enhances food preservation capability compared to refrigeration alone, or put another way, implementing ozone sanitation of foods in a refrigerator adds to the primary food preservation by inhibiting bacterial and fungal growth on food and vegetable surfaces.

The refrigerator sanitation system 600 may include an electro-ionic device 610 for emitting ozone in a refrigerator 602 and/or within one or more drawer compartments 604 within the refrigerator 602. The electro-ionic device 610 may be substantially the same as the electro-ionic device 110 or ozone generator 200 in that it includes the same subcomponents described above such as the emitter 126 and the collector 128 (although not shown in the electro-ionic device 610). The electro-ionic device 610 may be integrated into a wall of the refrigerator 602 or installed in the main compartment.

The refrigerator sanitation system 600 may include the controller 212 and one or more ozone sensors 210 to provide feedback for a controlled release of ozone in the refrigerator 602 or drawer compartment 604. For example, the controller 212 may be configured to maintain a predetermined concentration of ozone, such as a concentration between 0.05 to 0.15 ppm or less than 0.05 ppm. In other embodiments, the refrigerator sanitation system 600 may be configured to maintain the predetermined concentration of ozone without the use of one or more ozone sensors 210.

When the door of the refrigerator 602 is opened and closed, some of the cooled air escapes and is replaced with room temperature air. Depending on the climate and other factors, such as whether the air is conditioned, the room temperature air may contain more water vapor than inside of the refrigerator sanitation system. As the room temperature air cools to the inside temperature, water may condense on various surfaces inside the refrigerator 602 and the ozone from the electro-ionic device 610 may help prevent bacteria, including legionella from forming if the refrigerator were unexpectedly unplugged.

The refrigerator sanitation system 600 may be initially designed and fabricated to include the electro-ionic unit 610 or the electro-ionic unit 610 may be retrofitted into an existing refrigerator 602, i.e., as a bolt-on unit. When the electro-ionic unit 610 is bolted onto an existing refrigerator 602, it can electrically connect to the refrigerator circuit that activates the compressor or the fan. Although a conventional refrigerator 602 is shown in FIG. 21, in other embodiments, an entire wine cellar or wine aging facility may be treated with ozone, where cool temperatures are desired and unwanted mold and bacteria are typically present.

Although ozone may irritate the lungs and other biologic tissue at some concentrations, it may also be therapeutic. Indeed, it has been found that ozone introduced into the respiratory tract and/or the GI tract may help treat a COVID-19 infection. For therapy, in some cases the concentration of ozone may be below 0.1 ppm, such as 0.05 ppm, but in other cases it may exceed this level. For example, the concentration may be between 0.1 and 0.15 ppm, between 0.15 and 0.2 ppm, or above ppm.

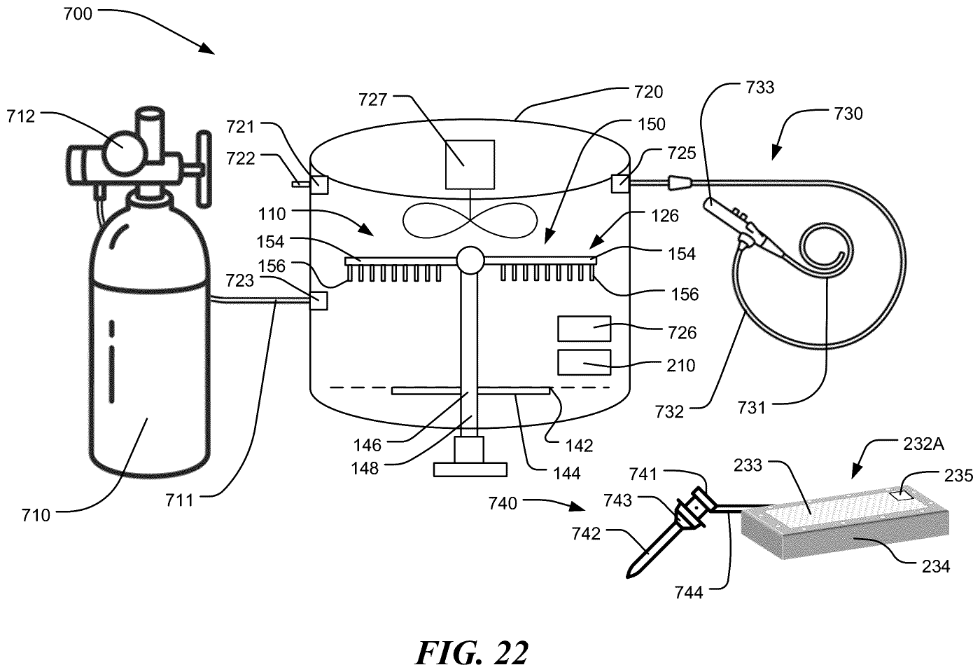

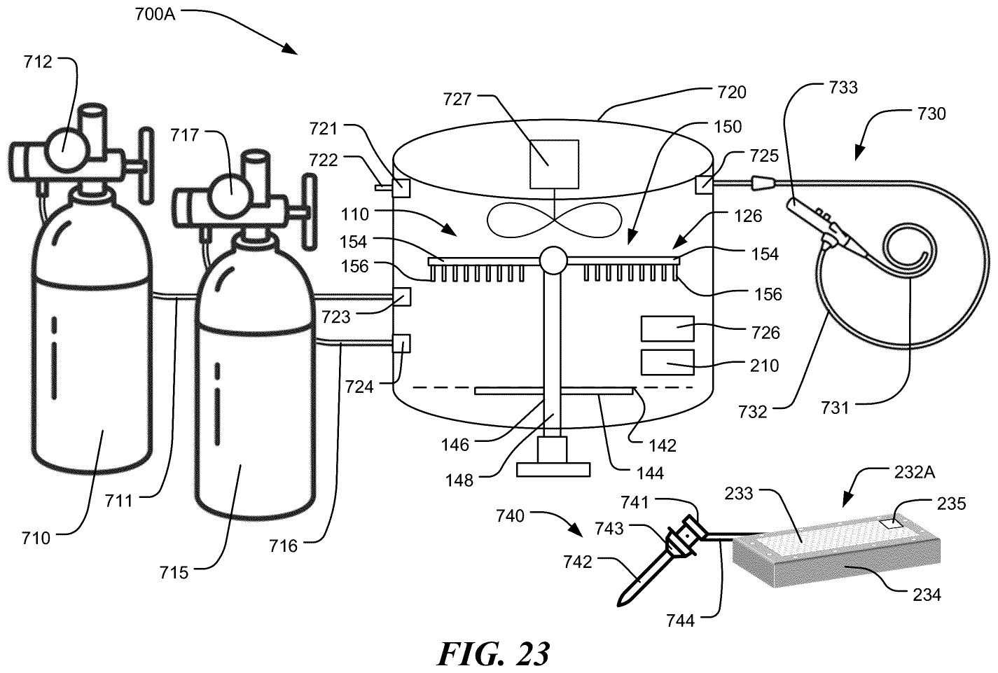

FIGS. 22 and 23 show exemplary insufflation systems 700 and 700A, respectively, configured to generate and introduce therapeutic ozone into the lower GI tract. Starting with FIG. 22, the insufflation system 700 may include a first raw medical gas source 710. The first raw gas source 710 may be a filled isolated gas tank located within an operating room or medical office, it may be a large gas tank configured to supply the first raw gas to an entire hospital or clinic, or it may be a compressor or other device configured to collect, purify, and/or concentrate the first raw gas. The first medical raw gas may include an oxygen gas source, such as pure or substantially pure oxygen (02), compressed air, carbon dioxide mixed with compressed air, carbon dioxide mixed with oxygen, or any other gas including at least oxygen in some concentration. The first raw gas source 710 may include tubing 711 to allow the first raw gas to flow to a chamber 720 for a portion of the oxygen contained therein to be converted to ozone. The first raw gas source 710 may also include a pressure regulator 712 to provide the operator precise control over the amount of gas entering the chamber 720. The pressure regulator 712 is shown diagrammatically as a mechanical pressure regulator, but in other embodiments the pressure regulator 712 may be a solenoid configured to provide precise automated electronic control of the gas flow to the chamber 720, such as a feedback controller discussed below in more detail.

The chamber 720 is diagrammatically shown as a cylinder, but may include substantially any geometry having an enclosed wall for containing a volume of gas, preferably pressurized above atmospheric pressure. The chamber 720 may include the electro-ionic device 110 as discussed above in more detail. In particular, the electro-ionic device 110 may be integrated into the chamber as a standalone device, or as a bolt-on or deployable device for retrofitting an existing chamber 720 in a similar manner as the electro-ionic device 110 described with regard to FIGS. 7-11B above. As discussed above, based on the applied voltage and gap spacing of emitter 156 and a dedicated collector plate 128 (not shown) or the internal surface of the chamber 720 acting as the collector, the amount of ozone generated can be optimized and controlled. The generated ozone may naturally diffuse throughout the chamber 720 via Brownian diffusion. In some embodiments, the chamber 720 may also include a fan 727 to help circulate and evenly diffuse the ozone generated therein.

The chamber 720 may include one or more openings controlled by solenoids or mechanical pressure regulators, such as solenoids 721, 723, and 725. As configured in the exemplary embodiment, the solenoid 723 may control the inlet pressure of the first raw gas entering the chamber 720 from the tubing 711 and the solenoid 725 may control outlet pressure of the ozonated gas leaving the chamber 720 through tubing 732. The solenoid 721 may permit the chamber 720 to purge gasses for various reasons, such as to help refresh and replenish the gas from any unintended secondary reactions of the ozone. A pressure sensor 726 and an ozone sensor 210 may monitor the pressure and ozone concentration, respectively, for feedback control of the ozone generation. For example, based on the pressure and the ozone concentration within the chamber 520, the applied voltage or duty cycle may be adjusted to produce more or less ozone or more of the first raw gas may be supplied to the chamber 720 to maintain ozone levels predetermined by the medical staff.

The tubing 732 directing the ozonated gas leaving the chamber 720 may be connected to an insufflation device, such as a dedicated insufflation device, colonoscope, endoscope, or the like. As shown in FIG. 22, the tubing 732 is connected to an instrument 730, such as a colonoscope, laparoscope, endoscope, thoracoscope, or any other scope, probe, catheter configured for introducing insufflation gas into the body. The instrument 730 may include a control interface 733 for the clinician to guide and control a distal end 731 of the instrument 730. The distal end 731 may have various ports, including a port to outlet the ozonated gas and optical guides and various optional items such as end effectors, electrosurgical tools, suction ports, etc.

The distal end 731 of the instrument may be configured to enter the opening of the body cavity directly or it may enter the body through a sealing device 740 such as a trocar, insufflation seal, or the like to maintain distention of the body tissue or organ. The sealing device 740 may have an opening 741 configured to receive the distal end 731 in a manner to allow the distal end 731 to articulate axially with respect to the opening 741 while maintaining a substantially gas-tight seal. Opposite from the opening 741 is an introducer 742, which may include a sharp hollow tube configured to puncture or penetrate through an opening in the body to advance the distal end 731 of the instrument 730 into the body. A plug section 743 extends and widens proximally from the introducer 742 toward the opening 741. The plug section 743 is configured to form a seal with the cavity opening and outer surface of the body and maintain the insufflation gas at a desired pressure.

The sealing device 740 may have tubing 744 and a valve (not shown) to bleed off or vent the insufflation gas. The tubing 744 may route the insufflation gas to an ozone decomposition device 232A substantially the same as ozone decomposition device 232 described above with regard to FIGS. 13A and 13B, except with possible different geometries and tubing connectors (not shown) to connect to the tubing 744. The ozone decomposition device 232A may further include an ozone sensor 235 to provide ozone measurements as the gas is released to the atmosphere. In other embodiments not shown, the ozone decomposition device 232A may additionally or alternatively include one or more particle filters, including fibrous filters, carbon filters, or an additional electro-ionic device 110 as discussed above. The ozone sensor 235 may also cooperate with the other sensors and solenoids to help provide feedback control of the ozone concentration in the exhausted insufflation gas. For example, it may be desirable to maintain the exhausted ozone concentration below 0.05 ppm, for example, when the generated ozone concentration exceeds it.

In another embodiment not shown, the sealing device 740 may include in an inner chamber having an ozone decomposition device comprised of the same materials as the ozone decomposition device 232 discussed above in a single unit without the need to route the insufflation gas to a separate unit.

During a procedure, such as, but not limited to a colonoscopy, endoscopy, laparoscopy, or thoracoscopy, the insufflation system 700 may be configured to output the ozonated gas up to 6 L/min and up to 8 psi, preferably between 1.5 L/min and 4 L/min and between 5.5 psi and 7.5 psi, while maintaining a target ozone concentration between 0.5 ppm and 2.5 ppm.

Turning to FIG. 23, another embodiment of an insufflation system 700A is shown. The insufflation system 700A is substantially the same as insufflation system 700 except that it may include a second raw gas source 715, and associated tubing 716, pressure regulator 717, and solenoid 724. Some existing insufflation systems may use a second raw gas source 715 comprising an inert gas or substantially inert gas such as carbon dioxide, nitrogen, helium, argon, or xenon. In these systems, the inert gas may not oxidize into ozone and, accordingly, an oxygen source may be necessary to generate ozone. Thus, the insufflation system 700A may also use the first raw gas source 710 including preferably, compressed air or oxygen. The first raw gas and the second raw gas may mix within the chamber 720 based on a predetermined pressure ratio. The pressure ratio between the first and second raw gasses may be adjusted based on the values measured at the pressure sensor 726 and the ozone sensor 210. The remaining operation of the insufflation system 700A may be substantially the same as the insufflation system 700 discussed above.

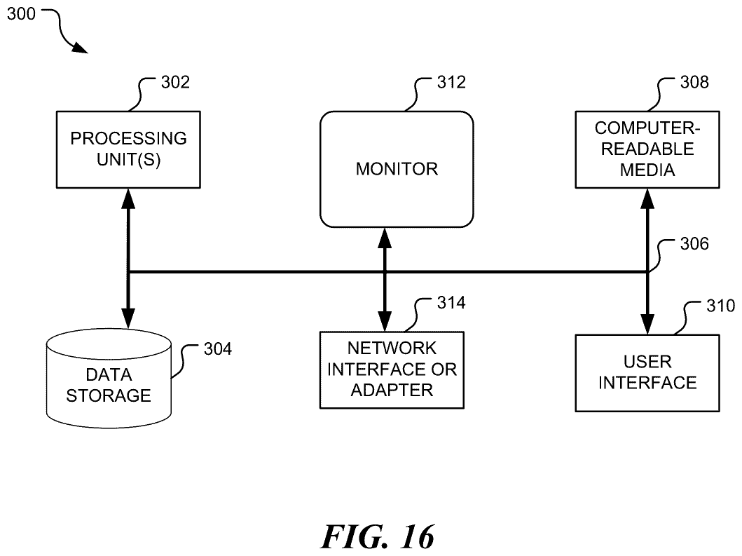

FIG. 16 illustrates an example of a suitable computing and networking environment 300 that may be used to implement various aspects of the present disclosure described herein and depicted in the various FIGS. of this patent disclosure. As illustrated, the computing and networking environment 300 includes a general purpose computing device 300 capable of operating the functions of the data logger, and/or the controller in FIG. 16, although it is contemplated that the networking environment 300 may include other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 300 may include various hardware components, such as a processing unit 302, a data storage 304 (e.g., a system memory), and a system bus 306 that couples various system components of the computer 300 to the processing unit 302. The system bus 306 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 300 may further include a variety of computer-readable media 308 that includes removable/non-removable media and volatile/nonvolatile media, but excludes transitory propagated signals. Computer-readable media 308 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 300. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 304 includes computer storage media in the form of volatile/nonvolatile memory such as read only memory (ROM) and random access memory (RAM). A basic input/output system (BIOS), containing the basic routines that help to transfer information between elements within the computer 300 (e.g., during start-up) is typically stored in ROM. RAM typically contains data and/or program modules that are immediately accessible to and/or presently being operated on by processing unit 302. For example, in one embodiment, data storage 304 holds an operating system, application programs, and other program modules and program data.

Data storage 304 may also include other removable/non-removable, volatile/nonvolatile computer storage media. For example, data storage 304 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 16, provide storage of computer-readable instructions, data structures, program modules and other data for the computer 300.

A user may enter commands and information through a user interface 310 or other input devices such as a tablet, electronic digitizer, a microphone, keyboard, and/or pointing device, commonly referred to as mouse, trackball or touch pad. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 302 through a user interface 310 that is coupled to the system bus 306, but may be connected by other interface and bus structures, such as a parallel port, game port or a universal serial bus (USB). A monitor 312 or other type of display device is also connected to the system bus 306 via an interface, such as a video interface. The monitor 312 may also be integrated with a touch-screen panel or the like.

The computer 300 may operate in a networked or cloud-computing environment using logical connections of a network interface or adapter 314 to one or more remote devices, such as a remote computer. The remote computer may be a personal computer, a server, a router, a network PC, a peer device or other common network node, and typically includes many or all of the elements described above relative to the computer 300. The logical connections depicted in FIG. 16 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 300 may be connected to a public and/or private network through the network interface or adapter 314. In such embodiments, a modem or other means for establishing communications over the network is connected to the system bus 306 via the network interface or adapter 314 or other appropriate mechanism. A wireless networking component including an interface and antenna may be coupled through a suitable device such as an access point or peer computer to a network. In a networked environment, program modules depicted relative to the computer 300, or portions thereof, may be stored in the remote memory storage device.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

1. A medical insufflation device for use on a patient body, the medical insufflation device comprising:
a chamber configured to receive a medical gas at least including oxygen;
an ozone generator in communication with the medical gas and configured to generate an ozonated medical gas by converting at least a portion of the oxygen in the medical gas into ozone;
an instrument configured to be introduced into the patient body, the instrument further configured to receive the ozonated medical gas from the chamber and convey the ozonated medical gas into the patient body; and
a controller configured to control the medical insufflation device such that the ozonated medical gas conveyed to the patient body by the instrument is at a targeted amount of ozone.

2. The medical insufflation device of claim 1, wherein the targeted amount of ozone is below 0.1 ppm.

3. The medical insufflation device of claim 1, wherein the targeted amount of ozone is between 0.1 and 0.15 ppm.

4. The medical insufflation device of claim 1, wherein the targeted amount of ozone, between 0.15 and 0.2 ppm.

5. The medical insufflation device of claim 1, wherein the targeted amount of ozone is above 0.2 ppm.

6. The medical insufflation device of claim 1, wherein the instrument includes at least one of a viewing instrument facilitating internal viewing within the patient body, a medical instrument for performing a surgical procedure, or a channel through which surgery is performed.

7. The medical insufflation device of claim 1, wherein at least a portion of the ozone generator is located in the chamber.

8. The medical insufflation device of claim 1, further comprising:
a pressure sensor configured to measure a gas pressure at least associated with the ozonated medical gas; and
an ozone sensor configured to measure an ozone concentration of the ozonated medical gas,
wherein the controller is configured to control the medical insufflation device based on measured values from the pressure sensor and ozone sensor such that the ozonated medical gas conveyed to the patient body by the instrument is at the targeted amount of ozone.

9. The medical insufflation device of claim 1, further comprising:
a pressure sensor configured to measure a gas pressure of the ozonated medical gas in the chamber;
an ozone sensor configured to measure an ozone concentration of the ozonated medical gas in the chamber;
a first solenoid configured to regulate a flow of the medical gas entering the chamber; and
a second solenoid configured to regulate a flow of the ozonated medical gas exiting the chamber,
wherein the controller is configured to control at least the first and second solenoids based on measured values from the pressure sensor and ozone sensor to provide a targeted amount of ozone leaving the chamber through the second solenoid.

10. The medical insufflation device of claim 9, wherein the targeted amount of ozone is below 0.1 ppm.

11. The medical insufflation device of claim 9, wherein the targeted amount of ozone is between 0.1 and 0.15 ppm.

12. The medical insufflation device of claim 9, wherein the targeted amount of ozone, between 0.15 and 0.2 ppm.

13. The medical insufflation device of claim 9, wherein the targeted amount of ozone is above 0.2 ppm.

14. The medical insufflation device of claim 1, wherein the ozone generator includes an electro-ionic device in communication with the medical gas and configured to generate the ozonated medical gas by converting the at least a portion of the oxygen in the medical gas into ozone.

15. The medical insufflation device of claim 14, wherein the electro-ionic device includes an emitter and a collector plate.

16. The medical insufflation device of claim 15, wherein the emitter includes an ionizer needle emitter with free ends coated with at least one of zinc, iridium, and/or tantalum.

17. The medical insufflation device of claim 15, wherein the emitter includes an ionizer needle emitter with free ends coated with carbon nanotubes.

18. The medical insufflation device of claim 14, wherein the electro-ionic device employs an operational voltage of 10 kV to 100 kV.

19. The medical insufflation device of claim 14, wherein the electro-ionic device has an air gap of 2 cm.

20. The medical insufflation device of claim 14, wherein the electro-ionic device employs an electric field strength of at least 5 kV/cm.

21. The medical insufflation device of claim 1, further comprising a vent attachable to the patient body and configured to vent the ozonated medical gas from the patient body.

22. The medical insufflation device of claim 21, wherein the vent includes an ozone decomposition element.

23. The medical insufflation device of claim 22, wherein the ozone decomposition element includes an ozone sensor providing ozone measurements as the vented medical gas is released to atmosphere.

24. The medical insufflation device of claim 22, wherein the ozone decomposition element acts to maintain the vented medical gas at an ozone concentration of below 0.05 ppm when the targeted amount of ozone is above 0.05 ppm.

25. The medical insufflation device of claim 1, wherein the medical insufflation device is configured to output the ozonated medical gas up to 6 L/min and up to 8 psi while maintaining a targeted ozone concentration between 0.5 ppm and 2.5 ppm.

26. The medical insufflation device of claim 1, wherein the medical insufflation device is configured to output the ozonated medical gas between 1.5 L/min and 4 L/min and between 5.5 psi and 7.5 psi, while maintaining a targeted ozone concentration between 0.5 ppm and 2.5 ppm.

27.-68. (canceled)
